(19) United States
(12) Patent Application Publication
(10) Pub. No.: US 2024/0130788 A1
(43) Pub. Date: Apr. 25, 2024

(54) MEDICAL DEVICES WITH PROTECTED LIGHT CONDUCTORS

(71) Applicant: GYRUS ACMI, INC. D/B/A OLYMPUS SURGICAL TECHNOLOGIES AMERICA, Westborough, MA (US)

(72) Inventors: Nikhil M. Murdeshwar, Maple Grove, MN (US); Sergey A. Bukesov, Acton, MA (US)

(21) Appl. No.: 18/164,191

(22) Filed: Feb. 3, 2023

Related U.S. Application Data
(60) Provisional application No. 63/267,581, filed on Feb. 4, 2022.

(51) Int. Cl. A61B 18/26

(52) U.S. Cl. CPC A61B 18/26; A61B 2018/263

(57) ABSTRACT

A device for performing a surgical procedure can comprise a shaft extending from a proximal portion to a distal portion, a light conductor extending at least partially through the shaft to be exposed at the distal portion, and a damage mitigator positioned to receive light from the light conductor to discharge the light from the device. A method of preventing damage to an optical fiber in a medical device having laser treatment capabilities can comprise emitting a laser beam from the optical fiber, fragmenting a biological stone with the laser beam, and mitigating damage to the optical fiber from fragmentation of the biological stone.

### CROSS-REFERENCE TO RELATED APPLICATION

This patent application claims the benefit of U.S. Provisional Patent Application No. 63/267,581, filed Feb. 4, 2022, which is incorporated by reference herein in its entirety.

### TECHNICAL FIELD

The present disclosure relates generally to medical devices and instruments configured to provide diagnostic and treatment operations. More specifically, the present disclosure relates to medical device systems comprising elongate bodies that can be inserted into incisions or openings in anatomy of a patient and then advanced to reach locations deep within anatomic passageways of the patient where the diagnostic and treatment operations can be performed.

### BACKGROUND

Endoscopes can be used for one or more of 1) providing passage of other devices, e.g., therapeutic devices or tissue collection devices, toward various anatomical portions, and 2) imaging of such anatomical portions. Such anatomical portions can include the gastrointestinal tract (e.g., esophagus, stomach, duodenum, pancreaticobiliary duct, intestines, colon, and the like), the renal area (e.g., kidney(s), ureter, bladder, urethra) and other internal organs (e.g., reproductive systems, sinus cavities, submucosal regions, respiratory tract), and the like.

Conventional endoscopes can be involved in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, and providing suction passageways for collecting fluids (e.g., saline or other preparations) and the like.

In conventional endoscopy, the distal portion of the endoscope can be configured for supporting and orienting another instrument, such as via steering and the use of an elevator. In some systems, two endoscopes can be configured to work together with a first endoscope guiding a second endoscope inserted therein with the aid of an elevator that can turn the second endoscope relative to the first endoscope. Such systems can be helpful in guiding endoscopes to anatomic locations within the body that are difficult to reach. For example, some anatomic locations can only be accessed with an endoscope after passage through a circuitous path sometimes involving sharp turns between different anatomic passageways.

One example of an endoscopic procedure is called an Endoscopic Retrograde Cholangio-Pancreatography, hereinafter “ERCP” procedures. In an ERCP procedure, an “auxiliary scope” (also referred to as daughter scope or cholangioscope) can be attached and advanced through the working channel of a “main scope” (also referred to as mother scope or duodenoscope). Once the auxiliary scope has reached the desired location, various procedures can be performed, sometimes involving the use of an additional instrument or device. For example, a tissue retrieval device inserted through the auxiliary scope can be used to remove biological matter or a laser device can be used to break-up biological stones.

### SUMMARY

The present inventors have recognized that problems or shortcomings can be associated with medical procedures that user laser energy to fragment various biological stones in particular applications. For example, fragmentation systems that utilize laser energy, such as Electrohydraulic Lithotripsy (EHL), typically require a laser module and a light conductor to convey laser energy from the laser module to a distal or working end of an instrument, e.g., an endoscope. In typical laser-based treatment systems, large and powerful laser modules can be required to generate energy suitable for fragmenting stones. For example, some laser fragmentation systems utilize laser energy suitable for fragmenting stones from the gallbladder and pancreas that can comprise globules of softer material, such as bile and cholesterol.

However, harder stones of the urinary system that comprise calcified masses can require a larger amount of energy to fragment. U.S. Pat. No. 10,646,276 to Fan et al., the contents of which are hereby incorporated by reference, describes the use of Holmium:YAG (Ho:YAG) laser lithotripsy with a laser light of 2170 nm wavelength to break kidney stones by photothermal effect. U.S. Pat. No. 9,259,231 to Navve et al., the contents of which are hereby incorporated by reference, discloses the use of fibers having diameters of 200, 270 or 365 μm in laser lithotripsy procedures of the kidney.

The present inventors have recognized that light conductors used in the laser fragmenting procedures can become damage during the performance of the procedure. For example, in a laser lithotripsy procedure, a laser beam can enter into fluid surrounding a stone in the anatomy. The fluid can comprise biological fluid or fluid introduced into the anatomy from a medical device during the procedure. Energy from the laser beam can enter the fluid and generate a shockwave that can transmit energy to the stone. One or more bursts of the laser beam can be used to break-up the stone so that the stone can be processed by the anatomy (e.g., metabolized, digested or passed through the GI tract) or collected with a tissue retriever device.

The present inventors have recognized that laser shockwave fragmentation of stones can potentially cause damage to the light-emitting fibers used to generate the laser beam in a plurality of different mechanism. 1) The shockwave itself can cause damage to the material of the light-emitting fiber. 2) Stone fragments can be directed toward and impact the light-emitting fiber to cause damage. 3) Stone fragments can become heated during the fragmentation process and stone fragments directed back to the light-emitting fiber can cause heat damage (e.g., “burn back”) to the light-emitting fiber. Furthermore, coatings applied to the light-emitting fibers can additionally be damaged by such occurrences.

These problems and other problems can be exacerbated and generated, respectively, when the distal end of the light-emitting fiber is not positioned a desirable distance from the stone. For example, when the end of the light-emitting fiber is held within a desirable range of distances from the stone, the shockwave has a proper length of the fluid media available to properly disperse before impacting the stone. If the distance between the end of the light-emitting fiber is too short, the shockwave does not have enough fluid to properly form, which can result in a “drilling” effect where only a small indentation is produced in the stone. Drilling effect can result in many more laser activations being required to adequately break-up a stone. If the distance between the end of the light-emitting fiber is too long, the shockwave can disperse, and a vapor bubble can form in front of the scope. In such situations, the stone can be unaffected, but stray laser energy may still be deflected back to the laser fiber.

The present disclosure can provide solutions to these and other problems, such as by providing laser-based fragmentation systems that include light-emitting fibers that are protected from potential damage from the laser energy, the shockwave and the stones. The light-emitting fibers can be provided with a protection device or a mitigating device that can 1) prevent the formation of potential damage-causing occurrences, 2) mitigate the effects of potential damage-causing occurrences, and 3) shield the light-emitting fiber from potential damage-causing occurrences. In the case of 1), the protection device can facilitate holding the light-emitting fiber a desirable distance from the stone. In the case of 2), the protection device can allow the light-emitting fiber to better withstand the effects of the potential damage-causing occurrences. In the case of 3), the protection device can deflect or disperse energy from the potential damage-causing occurrences to lessen any adverse effects.

In an example, a device for performing a surgical procedure can comprise a shaft extending from a proximal portion to a distal portion, a light conductor extending at least partially through the shaft to be exposed at the distal portion, and a damage mitigator positioned to receive light from the light conductor to discharge the light from the device.

In another example, a method of preventing damage to an optical fiber in a medical device having laser treatment capabilities can comprise emitting a laser beam from the optical fiber, fragmenting a biological stone with the laser beam, and mitigating damage to the optical fiber from fragmentation of the biological stone.

### BRIEF DESCRIPTION OF DRAWINGS

FIG. 1 is a schematic diagram of an endoscopy system comprising an imaging and control system and an endoscope, such as duodenoscope, with which the biological matter collection systems and devices of the present disclosure can be used.

FIG. 2 is a schematic diagram of the imaging and control system of FIG. 1 showing the imaging and control system connected to the endoscope.

FIG. 3A is a schematic top view of a distal portion of the endoscope of FIG. 2 comprising a camera module including optical components for a side-viewing endoscope and an elevator mechanism.

FIG. 3B is an enlarged cross-sectional view taken along the plane 3B-3B of FIG. 3A showing the optical components.

FIG. 3C is an enlarged cross-sectional view taken along the plane 3C-3C of FIG. 3A showing the elevator mechanism.

FIG. 4 is a schematic illustration of a distal portion of an endoscope being used to position an auxiliary scope proximate a duodenum, the auxiliary scope being configured to receive a tissue retrieval device, including a tethered biopsy instrument, of the present disclosure.

FIG. 5A is a schematic illustration of a tissue retrieval device of the present disclosure comprising an elongate shaft and a translucent tissue collector.

FIG. 5B is a close-up view of a distal end of the tissue retrieval device of FIG. 5A showing the translucent tissue collector disposed within an auxiliary endoscope.

FIG. 6A is a schematic illustration of a translucent tissue collector comprising forceps in a closed state.

FIG. 6B is a schematic illustration of the translucent tissue collector of FIG. 6A with the forceps in an open state.

FIG. 7 is a schematic diagram illustrating a length of a light-conducting element with slack for a laser lithotripsy system extending through a shaft located between a proximal controller and a distal end portion.

FIG. 8A is a schematic cross-sectional view of a scope shaft comprising a plurality of elements extending through a tubular sheath including a loosely coiled light-conducting element.

FIG. 8B is a schematic side view of the scope of FIG. 8A showing the loosely coiled light conducting element wrapped around other elements.

FIG. 9A is a schematic cross-sectional view of a scope shaft comprising a plurality of lumens through which a plurality of elements extend including a loosely disposed light-conducting element.

FIG. 9B is a schematic side view of the scope of FIG. 9A showing the loosely disposed light-conducting element disposed within a slack chamber.

FIG. 10 is a schematic illustration of a distal end of a scope shaft having an integrated fiber that is recessed from a distal end surface of the scope shaft.

FIG. 11 is a schematic illustration of a distal end of a scope shaft having a prism disposed at a distal end of an integrated fiber.

FIG. 12 is a schematic illustration of a distal end of a light-conducting fiber having a shield placed at a distal tip of the light-conducting fiber.

FIG. 13A is a schematic illustration of a distal end of a light-conducting fiber having a bulbous tip geometry.

FIG. 13B is a schematic illustration of a distal end of a light-conducting fiber having a triangular-shaped tip geometry.

FIG. 13C is a schematic illustration of a distal end of a light-conducting fiber having a square-shaped tip geometry.

FIG. 13D is a schematic illustration of a distal end of a light-conducting fiber having a trapezoid-shaped tip geometry.

FIG. 14 is a schematic illustration of a waveform for generating a laser beam that can be used to dislodge debris from a discharge end of a scope.

### DETAILED DESCRIPTION

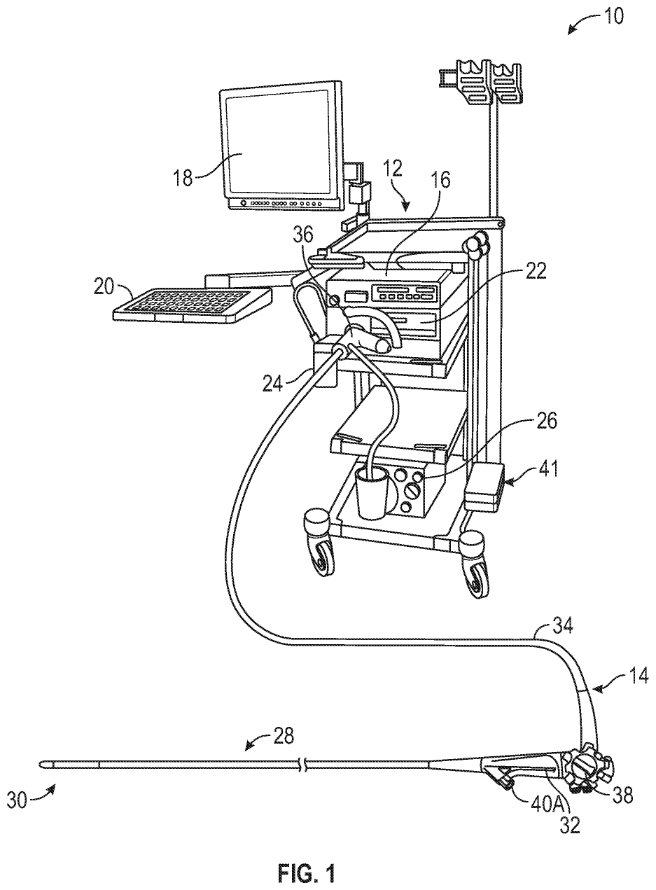

FIG. 1 is a schematic diagram of endoscopy system 10 comprising imaging and control system 12 and endoscope 14. The system of FIG. 1 is an illustrative example of an endoscopy system suitable for use with the systems, devices and methods described herein, such as laser-emitting devices having fragmentation energy, e.g., burn back, mitigating and preventing devices. According to some examples, endoscope 14 can be insertable into an anatomical region for imaging and/or to provide passage of or attachment to (e.g., via tethering) one or more sampling devices for biopsies, or one or more therapeutic devices for treatment of a disease state associated with the anatomical region. Endoscope 14 can, in advantageous aspects, interface with and connect to imaging and control system 12. In the illustrated example, endoscope 14 comprises a duodenoscope, though other types of endoscopes can be used with the features and teachings of the present disclosure. For example, endoscope 14 can comprise a cystoscope, ureteroscope, renoscope, nephroscope, or cholangioscope.

Imaging and control system 12 can comprise control unit 16, output unit 18, input unit 20, light source 22, fluid source 24 and suction pump 26.

Imaging and control system 12 can include various ports for coupling with endoscopy system 10. For example, control unit 16 can include a data input/output port for receiving data from and communicating data to endoscope 14. Light source 22 can include an output port for transmitting light to endoscope 14, such as via a fiber optic link. Fluid source 24 can include a port for transmitting fluid to endoscope 14. Fluid source 24 can comprise a pump and a tank of fluid or can be connected to an external tank, vessel or storage unit. Suction pump 26 can comprise a port used to draw a vacuum from endoscope 14 to generate suction, such as for withdrawing fluid from the anatomical region into which endoscope 14 is inserted. Output unit 18 and input unit 20 can be used by an operator of endoscopy system 10 to control functions of endoscopy system 10 and view output of endoscope 14. Control unit 16 can additionally be used to generate signals or other outputs from treating the anatomical region into which endoscope 14 is inserted. In examples, control unit 16 can generate electrical output, acoustic output, a fluid output and the like for treating the anatomical region with, for example, cauterizing, cutting, freezing and the like.

Endoscope 14 can comprise insertion section 28, functional section 30 and handle section 32, which can be coupled to cable section 34 and coupler section 36. Coupler section 36 can be connected to control unit 16 to connect to endoscope 14 to multiple features of control unit 16, such as input unit 20, light source unit 22, fluid source 24 and suction pump 26.

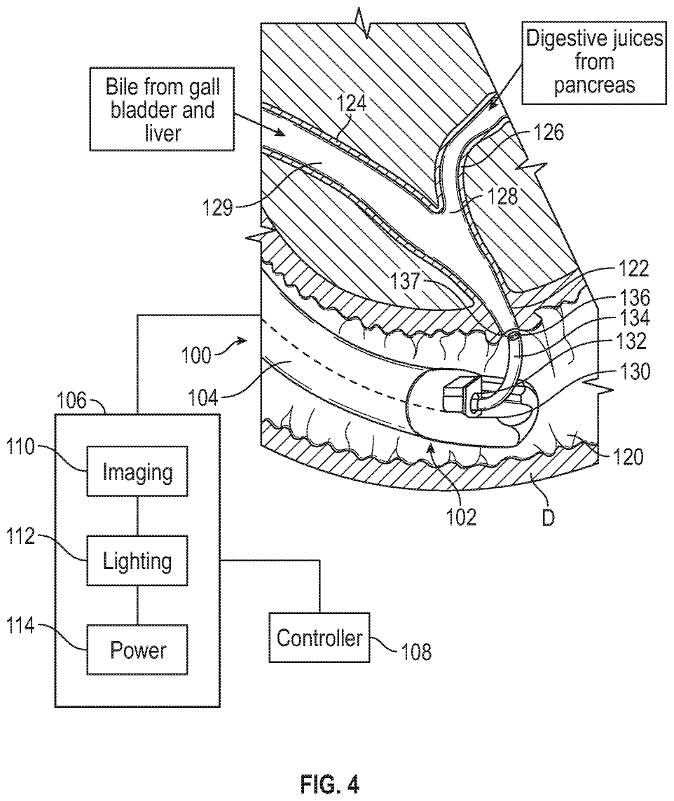

Insertion section 28 can extend distally from handle section 32 and cable section 34 can extend proximally from handle section 32. Insertion section 28 can be elongate and include a bending section, and a distal end to which functional section 30 can be attached. The bending section can be controllable (e.g., by control knob 38 on handle section 32) to maneuver the distal end through tortuous anatomical passageways (e.g., stomach, duodenum, kidney, ureter, etc.). Insertion section 28 can also include one or more working channels (e.g., an internal lumen) that can be elongate and support insertion of one or more therapeutic tools of functional section 30, such as auxiliary scope 134 of FIG. 4. The working channel can extend between handle section 32 and functional section 30. Additional functionalities, such as fluid passages, guide wires, and pull wires can also be provided by insertion section 28 (e.g., via suction or irrigation passageways, and the like).

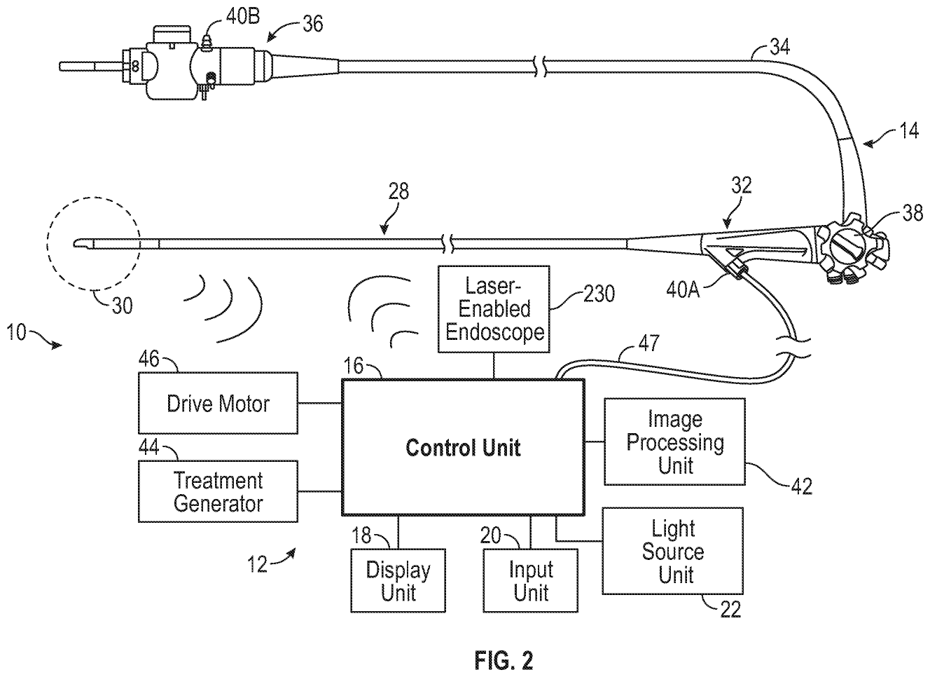

Handle section 32 can comprise knob 38 as well as port 40A. Knob 38 can be coupled to a pull wire, or other actuation mechanisms, extending through insertion section 28. Port 40A, as well as other ports, such as port 40B (FIG. 2), can be configured to couple various electrical cables, guide wires, auxiliary scopes, tissue collection devices of the present disclosure, fluid tubes and the like to handle section 32 for coupling with insertion section 28.

Imaging and control system 12, according to examples, can be provided on a mobile platform (e.g., cart 41) with shelves for housing light source 22, suction pump 26, image processing unit 42 (FIG. 2), etc. Alternatively, several components of imaging and control system 12 shown in FIGS. 1 and 2 can be provided directly on endoscope 14 so as to make the endoscope “self-contained.”

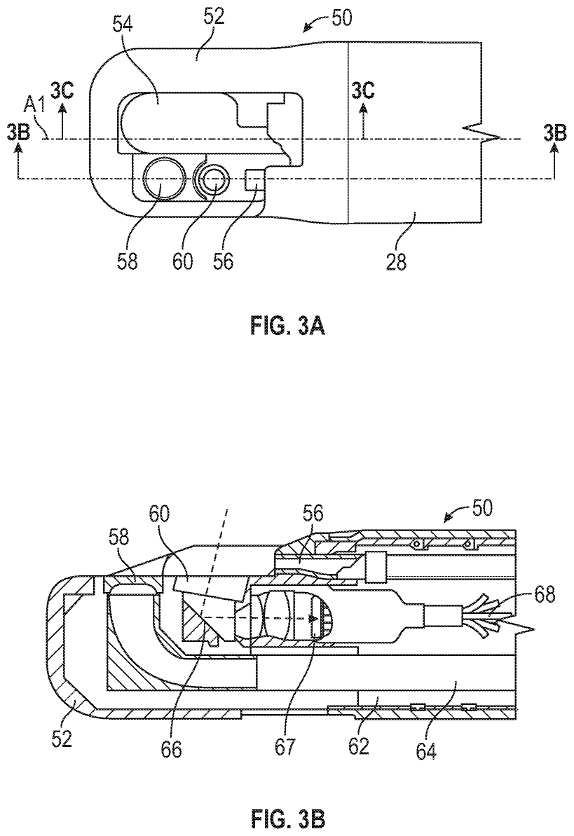

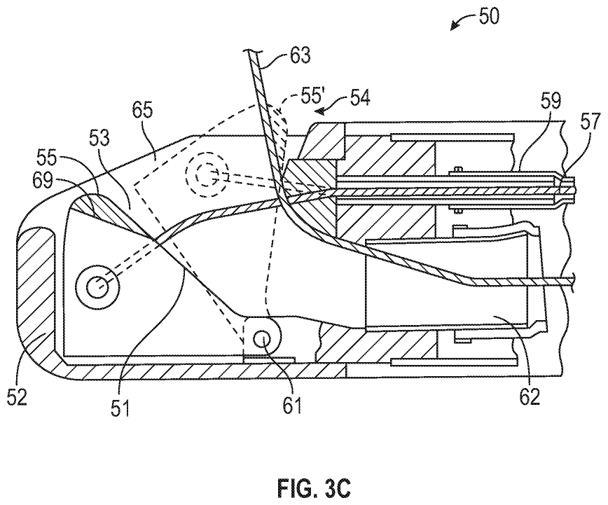

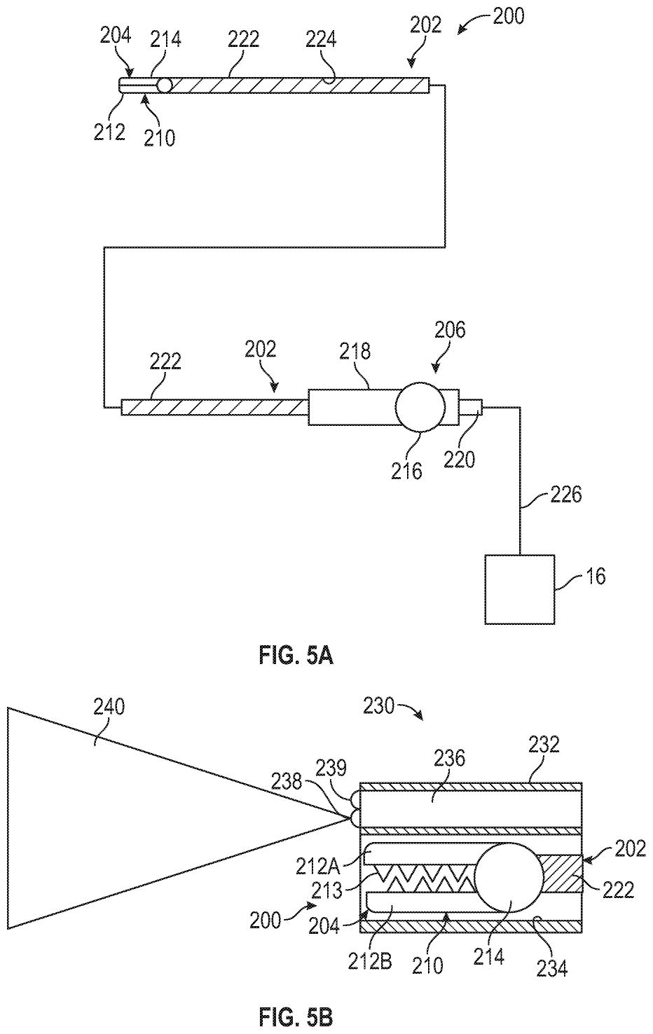

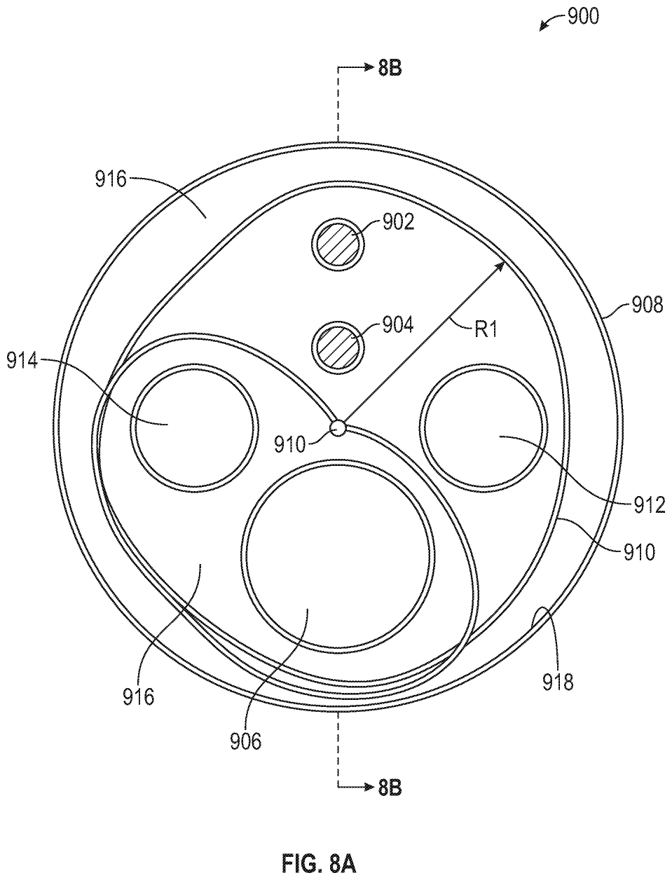

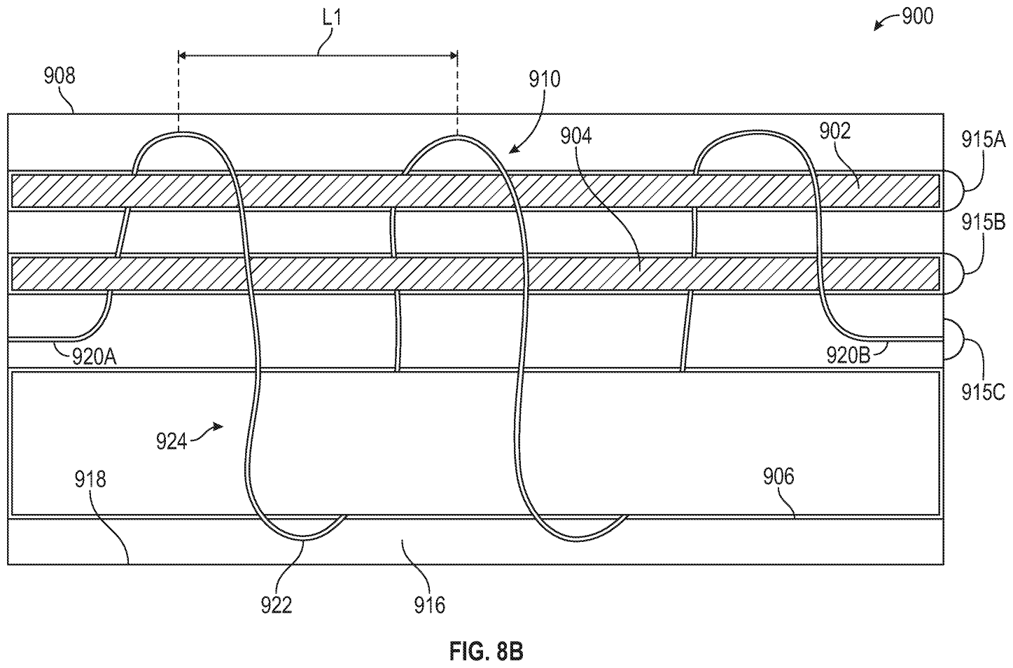

Functional section 30 can comprise components for treating and diagnosing anatomy of a patient. Functional section 30 can comprise an imaging device, an illumination device and an elevator, such as is described further with reference to elevator 54 of FIGS. 3A-3C. Functional section 30 can further comprise optically enhanced biological matter and tissue collection and retrieval devices as are described herein. For example, functional section 30 can comprise one or more electrodes conductively connected to handle section 32 and functionally connected to imaging and control system 12 to analyze biological matter in contact with the electrodes based on comparative biological data stored in imaging and control system 12. In other examples, functional section 30 can directly incorporate tissue collectors similar to the tissue retrieval devices described with reference to FIGS. 5A-7B and the biopsy devices described with reference to FIGS. 8A-11.

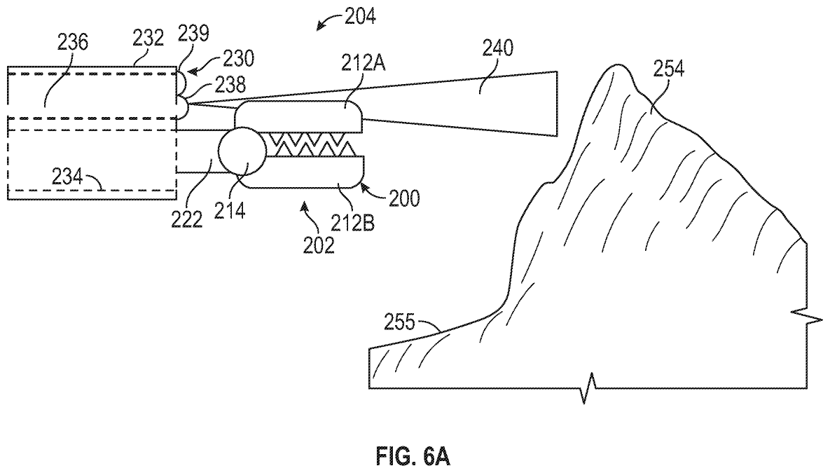

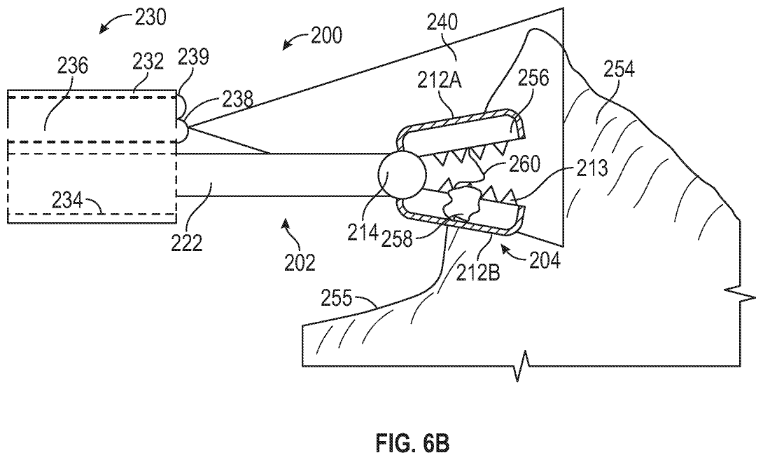

FIG. 2 is a schematic diagram of endoscopy system 10 of FIG. 1 comprising imaging and control system 12 and endoscope 14. FIG. 2 schematically illustrates components of imaging and control system 12 coupled to endoscope 14, which in the illustrated example comprises a duodenoscope. Imaging and control system 12 can comprise control unit 16, which can include or be coupled to image processing unit 42, treatment generator 44 and drive unit 46, as well as light source 22, input unit 20 and output unit 18. Coupler section 36 can be connected to control unit 16 to connect to endoscope 14 to multiple features of control unit 16, such as image processing unit 42 and treatment generator 44. In examples, port 40A can be used to insert another instrument or device, such as a daughter scope or auxiliary scope, into endoscope 14. Such instruments and devices can be independently connected to control unit 16 via cable 47. In examples, port 40B can be used to connect coupler section 36 to various inputs and outputs, such as video, air, light and electric. As is discussed below in greater detail with reference to FIGS. 4-6B, control unit 16 can comprise, or can be in communication with, endoscope 100, surgical instrument 200, which can comprise a device configured to engage tissue and collect and store a portion of that tissue and through which imaging equipment (e.g., a camera) can view target tissue via inclusion of optically enhanced materials and components, and endoscope 230, which can comprise a laser-emitting device configured to fragment stones. Control unit 16 can be configured to activate a camera to view target tissue distal of surgical instrument 200, which can be fabricated of a translucent material to minimize the impacts of the camera being obstructed or partially obstructed by the tissue retrieval device. Likewise, control unit 16 can be configured to activate light source 22 to shine light on surgical instrument 200, which can include select components that are configured to reflect light in a particular manner, such as tissue cutters being enhanced with reflective particles. Furthermore, control unit 16 can be configured to activate a laser generator for endoscope 230 (FIGS. 6A and 6B) to provide stone fragmenting energy to light conducting fibers of the present disclosure.

Image processing unit 42 and light source 22 can each interface with endoscope 14 (e.g., at functional unit 30) by wired or wireless electrical connections. Imaging and control system 12 can accordingly illuminate an anatomical region, collect signals representing the anatomical region, process signals representing the anatomical region, and display images representing the anatomical region on display unit 18. Imaging and control system 12 can include light source 22 to illuminate the anatomical region using light of desired spectrum (e.g., broadband white light, narrow-band imaging using preferred electromagnetic wavelengths, and the like). Imaging and control system 12 can connect (e.g., via an endoscope connector) to endoscope 14 for signal transmission (e.g., light output from light source, video signals from imaging system in the distal end, diagnostic and sensor signals from a diagnostic device, and the like).

Fluid source 24 (FIG. 1) can be in communication with control unit 16 and can comprise one or more sources of air, saline or other fluids, as well as associated fluid pathways (e.g., air channels, irrigation channels, suction channels) and connectors (barb fittings, fluid seals, valves and the like). Fluid source 24 can be utilized as an activation energy for a biasing device or a pressure-applying device of the present disclosure. Imaging and control system 12 can also include drive unit 46, which can be an optional component. Drive unit 46 can comprise a motorized drive for advancing a distal section of endoscope 14, as described in at least PCT Pub. No. WO 2011/140118 A1 to Frassica et al., titled “Rotate-to-Advance Catheterization System,” which is hereby incorporated in its entirety by this reference.

FIGS. 3A-3C illustrate a first example of functional section 30 of endoscope 14 of FIG. 2. FIG. 3A illustrates a top view of functional section 30. FIG. 3B illustrates a cross-sectional view of functional section 30 taken along section plane 3B-3B of FIG. 3A. FIG. 3C illustrates a cross-sectional view of functional section 30 taken along section plane 3C-3C of FIG. 3A.

FIGS. 3A-3C illustrate “side-viewing endoscope” (e.g., duodenoscope) camera module 50. In side-viewing endoscope camera module 50, illumination and imaging systems are positioned such that the viewing angle of the imaging system corresponds to a target anatomy lateral to central longitudinal axis A1 of endoscope 14. However, the biological matter retrieval devices can be used with other types of endoscopes, such as “end-viewing endoscopes.”

In the example of FIGS. 3A and 3B, side-viewing endoscope camera module 50 can comprise housing 52, elevator 54, fluid outlet 56, illumination lens 58 and objective lens 60. Housing 52 can form a fluid tight coupling with insertion section 28. Housing 52 can comprise opening for elevator 54. Elevator 54 can comprise a mechanism for moving a device inserted through insertion section 28, such as auxiliary scope 134 of FIG. 4. In particular, elevator 54 can comprise a device that can bend an elongate device extended through insertion section 28 along axis A1, as is discussed in greater detail with reference to FIG. 3C. Elevator 54 can be used to bend the elongate device at an angle to axis A1 to thereby treat or access the anatomical region adjacent side-viewing endoscope camera module 50. Elevator 54 is located alongside, e.g., radially outward of axis A1, illumination lens 58 and objective lens 60.

As can be seen in FIG. 3B, insertion section 28 can comprise central lumen 62 through which various components (e.g., auxiliary scope 134 (FIG. 4) can be extended to connect functional section 30 with handle section 32 (FIG. 2). For example, illumination lens 58 can be connected to light transmitter 64, which can comprise a fiber optic cable or cable bundle extending to light source 22 (FIG. 1). Likewise, objective lens 60 can be coupled to prism 66 and imaging unit 67, which can be coupled to wiring 68. Also, fluid outlet 56 can be coupled to fluid line 69, which can comprise a tube extending to fluid source 24 (FIG. 1). Other elongate elements, e.g., tubes, wires, cables, can extend through lumen 62 to connect functional section 30 with components of endoscopy system 10, such as suction pump 26 (FIG. 1) and treatment generator 44 (FIG. 2).

FIG. 3C a schematic cross-sectional view taken along section plane 3C-3C of FIG. 3A showing elevator 54. Elevator 54 can comprise deflector 55 that can be disposed in space 53 of housing 52. Deflector 55 can be connected to wire 57, which can extend through tube 59 to connect to handle section 32. Wire 57 can be actuated, such as by rotating a knob, pulling a lever, or pushing a button on handle section 32. Movement of wire 57 can cause rotation, e.g., clockwise, from a first position of deflector 55 about pin 61 to a second position of deflector 55, indicated by 55′. Deflector 55 can be actuated by wire 57 to move the distal portion of instrument 63 extending through window 65 in housing 52.

Housing 52 can comprise accommodation space 53 that houses deflector 55. Instrument 63 can comprise forceps, a guide wire, a catheter, or the like that extends through lumen 62. Instrument 63 can additionally comprise auxiliary scope 134 of FIG. 4, or a tissue collection device such as surgical instrument 200 of FIGS. 5A-6B. A proximal end of deflector 55 can be attached to housing 62 at pin 61 provided to the rigid tip 21. A distal end of deflector 55 can be located below window 65 within housing 62 when deflector 55 is in the lowered, or un-actuated, state. The distal end of deflector 55 can at least partially extend out of window 65 when deflector 55 is raised, or actuated, by wire 57. Instrument 63 can slide on angled ramp surface 51 of deflector 55 to initially deflect the distal end of instrument 63 toward window 65. Angled ramp surface 51 can facilitate extension of the distal portion of instrument 63 extending from window 65 at a first angle relative to the axis of lumen 62. Angled ramp surface 51 can include groove 69, e.g. a v-notch, to receive and guide instrument 63. Deflector 55 can be actuated to bend instrument 63 at a second angle relative to the axis of lumen 62, which is closer to perpendicular that the first angle. When wire 57 is released, deflector 55 can be rotated, e.g., counter-clockwise, back to the lowered position, either by pushing or relaxing of wire 57. In examples, instrument 63 can comprise a cholangioscope or auxiliary scope 134 (FIG. 4).

Side-viewing endoscope camera module 50 of FIGS. 3A-3C can include optical components (e.g., objective lens 60, prism 66, imaging unit 67, wiring 68) for collection of image signals, lighting components (e.g., illumination lens 58, light transmitter 64) for transmission or generation of light. Endoscope camera module 50 can also include a photosensitive element, such as a charge-coupled device (“CCD” sensor) or a complementary metal-oxide semiconductor (“CMOS”) sensor. In either example, imaging unit 67 can be coupled (e.g., via wired or wireless connections) to image processing unit 42 (FIG. 2) to transmit signals from the photosensitive element representing images (e.g., video signals) to image processing unit 42, in turn to be displayed on a display such as output unit 18. In various examples, imaging and control system 12 and image processing unit 67 can be configured to provide outputs at desired resolution (e.g., at least 480p, at least 720p, at least 1080p, at least 4K UHD, etc.) suitable for endoscopy procedures.

Thus, as endoscope 100 is inserted further into the anatomy, the complexity with which it must be maneuvered and contorted increases, as described with reference to FIG. 4. Furthermore, in order to reach locations even further in the anatomy, additional devices can be used, e.g., instrument 63 in the form of auxiliary scope 134. As such, the cross-sectional area, e.g., diameter, of subsequently nested devices becomes smaller, thereby requiring even smaller devices that can be difficult to manufacture and manipulate, or satisfactorily produce results without repeated interventions (e.g., interactions with the patient), as is described with reference to FIGS. 5A-7B.

FIG. 4 is a schematic illustration of distal portion of endoscope 100 according to the present disclosure positioned in duodenum D. Endoscope 100 can comprise functional module 102, insertion section module 104, and control module 106. Control module 106 can include controller 108, which can comprise a user input device configured to operate one or both of endoscope 100 and auxiliary scope 134. Control module 106 can include other components, such as those described with reference to endoscopy system 10 (FIG. 1) and control unit 16 (FIG. 2). Additionally, control module 106 can comprise components for controlling a camera and a light source connected to auxiliary scope 134, such as imaging unit 110, lighting unit 112 and power unit 114. Endoscope 100 can be configured similarly as endoscope 14 of FIGS. 1 and 2.

Duodenum D can comprise duct wall 120, sphincter of Oddi 122, common bile duct 124 and main pancreatic duct 126. Duodenum D comprises an upper part of the small intestine. Common bile duct 124 carries bile from the gallbladder and liver (not illustrated) and empties the bile into the duodenum D through sphincter of Oddi 122. Main pancreatic duct 126 carries pancreatic juice from the exocrine pancreas (not illustrated) to common bile duct 124. Sometimes it can be desirable to remove biological matter, e.g., tissue, from bile duct 124 or pancreatic duct 126 to analyze the tissue to, for example, diagnose diseases or maladies of the patient such as cancer.

Functional module 102 can comprise elevator portion 130. Endoscope 100 can further comprise lumen 132 and auxiliary scope 134. Auxiliary scope 134 can comprise lumen 136. Auxiliary scope 134 can itself include functional components, such as camera lens 137 and a light lens (not illustrated) coupled to control module 106, to facilitate navigation of auxiliary scope 134 from endoscope 100 through the anatomy and to facilitate viewing of components extending from lumen 132.

In certain duodenoscopy procedures (e.g., Endoscopic Retrograde Cholangio-Pancreatography, hereinafter “ERCP” procedures) an auxiliary scope (also referred to as daughter scope, or cholangioscope), such as auxiliary scope 134, can be attached and advanced through lumen 132 (or central lumen 62 of insertion section 28 of endoscope 14 in FIG. 3B) of the “main scope” (also referred to as mother scope, or duodenoscope), such as endoscope 100. As discussed in greater detail below, auxiliary scope 134 can be guided into sphincter of Oddi 122. Therefrom, a surgeon operating auxiliary scope 134 can navigate auxiliary scope 134 through lumen 132 toward the gall bladder, liver or other locations in the gastrointestinal system to perform various procedures. The surgeon can navigate auxiliary scope 134 past entry 128 of main pancreatic duct 126 and into passage 129 of common bile duct 124, or into entry 128. Auxiliary scope 134 can be used to guide an additional device to the anatomy to obtain biological matter, such as by passage through or attachment to lumen 136. The additional device can have its own functional devices, such as a light source, camera, tissue separators, accessories, and biopsy channel, for therapeutic procedures. As described with reference to FIGS. 5A-6B, the additional device can include various features, such as forceps, for gathering biological matter, such as tissue, and laser capabilities for fragmenting stones. In examples, the biological matter can then be removed from the patient, typically by removal of the additional device from the auxiliary device, so that the removed biological matter can be analyzed to diagnose one or more conditions of the patient. According to several examples, endoscope 100 can be suitable for the removal of cancerous or pre-cancerous matter (e.g., carcinoma, sarcoma, myeloma, leukemia, lymphoma and the like), endometriosis evaluation, biliary ductal biopsies, and the like.

However, as mentioned above, the size of the additional device is typically small due to the progressively smaller sizes of endoscope 100, auxiliary scope 134 and the additional device. In examples, lumen 132 of endoscope 100 can typically be on the order of approximately 4.0 mm in diameter, while lumen 136 of auxiliary scope 134 can typically be on the order of approximately 1.2 mm. As such, with conventional devices, it can be difficult to obtain sufficiently large tissue sample sized to ensure accurate diagnoses without having to repeatedly remove and reinsert the additional device. Likewise, it can be difficult to view the desired matter, e.g., the target tissue, due to multiple reasons including the presence of the tissue retrieval device in the line of sight of the auxiliary scope camera. This thereby makes collection of non-desirable, e.g., non-cancerous, material a possibility. However, with the systems and devices of the present disclosure it is possible to obtain sufficiently large tissue sample sizes with only a single insertion and removal of the additional device, when configured as a tissue retrieval device or biopsy instrument of the present disclosure, for example. For example, the tissue retrieval device can be fabricated partially or entirely of translucent materials to allow imaging devices to have improved visibility of tissue behind the tissue retrieval device. Additionally, the tissue retrieval device can be fabricated partially or entirely of reflective materials to allow imaging devices to have improved visibility of particular components, e.g., functional components such as tissue cutters, of the tissue retrieval device.

FIG. 5A is a schematic illustration of surgical instrument 200 comprising elongate body 202, tissue collection device 204 and device controller 206. Surgical instrument 200 can comprise a device configured for the separation, collection and retrieval of biological matter, such as tissue, from a patient. Tissue collection device 204 can comprise separator 210, which, in the illustrated example, comprises jaws 212 and hinge 214 and activation mechanism 216. Controller 206 can comprise handpiece or handle 218, which can include activation mechanism 216 and connector 220. Elongate body 202 can comprise shaft 222 that can include lumen 224. Controller 206 can be connected to system control unit 16 (FIGS. 1 and 2) via cable 226 and the use of connector 220. The components illustrated in FIGS. 5A and 5B are not necessarily drawn to scale.

Tissue collection device 204 can be configured to do one or both of separate and retrieve biological matter from within a patient after being positioned within the patient by elongate body 202. Tissue collection device 204 can be configured to engage target tissue, separate the target tissue from the patient and store separated target tissue for removal from the patient, such as by removal of elongate body 202 from the patient. The terms “tissue retrieval device” and “biopsy instrument” are used throughout the present disclosure, however a tissue retrieval device or biopsy instrument can alternatively or additionally comprise a biological matter collection device, a biological matter retrieval device, a tissue collection device and tissue retrieval device.

Handpiece 218 can comprise any device suitable for facilitating manipulation and operation of surgical instrument 200. Handpiece 218 can be located at the proximal end of shaft 222 or another suitable location along shaft 222. In examples, handpiece 218 can comprise a pistol grip, a knob, a handlebar grip and the like. Actuation mechanism 216 can be attached to handpiece 218 to operate tissue collection device 204. Actuation mechanism 216 can comprise one or more of buttons, triggers, levers, knobs, dials and the like. As such, actuation mechanism 216 can comprise a linkage located within lumen 224 of shaft 222 or alongside shaft 222. In examples, the linkage can be a mechanical linkage, an electronic linkage or an electric linkage, (such as a wire or cable), or an activation energy source, such as an electric source, a fluid source or a gas source (such as a tube or conduit).

Shaft 222 can extend from handpiece 218 and can comprise an elongate member configured to allow tissue collection device 204 to be inserted into a patient. In examples, shaft 222 can be sized for placement within an auxiliary scope, such as scope 134 of FIG. 4. As such, shaft 222 can be inserted into an incision in the epidermis of a patient, through a body cavity of the patient and into an organ. Thus, it is desirable for the diameter or cross-sectional shape of shaft 222, as well as components attached thereto, to be as small as possible to facilitate minimally invasive surgical procedures. Tissue collection device 204 can thus be incorporated into shaft 222 to minimize the size impact on surgical instrument 200 and without interfering with the linkage. Shaft 222 can be axially rigid, but resiliently bendable, and formed from a metal or plastic material.

Tissue collection device 204 can be located at the distal end of shaft 222 or another suitable location along shaft 222. Tissue collection device 204 can be sized to fit within lumen 136 (FIG. 4), for example. Tissue collection device 204 can comprise a component or device for interacting with a patient, such as those configured to cut, slice, pull, saw, punch, twist or auger tissue, and the like. Specifically, separator 210 can comprise any device suitable for removing tissue from a patient, such as a blade, punch, scraping device or an auger. In additional examples, jaws 212 can comprise a device configured to scrape or abrade tissue from the patient, such as a brush or grater device. In another example, jaws 212 can comprise a roughened surface, such as a surface coated with hard particles, such as diamond or sand particles. Separator 210 can be configured to physically separate portions of tissue of a patient from other larger portions of tissue in the patient. In additional examples, separator 210 can be configured to simply collect biological matter from the patient that does not need physical separation, such as mucus or fluid. In examples, jaws 212 can be configured to physically separate portion of tissue of a patient for retrieval with the tissue collection device or another device. In the illustrated example, separator 210 can comprise forceps having jaws 212 pivotably connected at hinge 214. Separator 210 can, however, be configured as a variety of devices capable of collecting biological matter, such as a punch, an auger, a blade, a saw and the like. Likewise, separator 210 can incorporate features for storing collected matter, such as a container or storage space. In an example, as discussed with reference to FIG. 6A, the storage space can be provided between jaws. Separator 210 can comprise forceps, as is described with reference to FIGS. 6A and 6B. In any configuration, portions of separator 210 can be configured to allow light to pass therethrough or to reflect light incident thereon to selectively enhance images of separator 210 and anatomy obtained by an imaging unit.

Jaws 212 can be configured as a container or a walled element to hold and retain biological matter collected by tissue collection device 204. In an example, jaws 212 can comprise a flexible basket that can be deformed to allow portions of jaws 212 to be brought into close contact with target tissue. For example, jaws 212 can be fabricated from woven material such as strands of Kevlar, PVC, polyethylene, polycarbonate, PEEK and the like. Jaws 212 can be coupled to structural components, e.g., a frame, to facilitate coupling to shaft 222 and to facilitate mounting of cutting elements, such as teeth or blades, to jaws 212, as well as to provide stability for separator 210. In additional examples, jaws 212 can comprise a structural element, such as a box fabricated from rigid and inflexible material.

Handpiece 218 can be operated by a user to operate tissue collection device 204. Handpiece 218 can be used to manipulate shaft 222 to push separator 210 against target tissue. For example, shaft 222 can be rotated, oscillated, reciprocated and the like move separator 210 along the target tissue to cause separator 210 to separate sample tissue from the target tissue attached to the patient. Activation mechanism 216 can be coupled to handpiece 218 and can be configured to operate separator 210. Activation mechanism 216 can comprise any type of device suitable for activating the different types of separator devices described herein. In examples, activation mechanism 216 can comprise one or more of a lever, a trigger, a joystick, a button, a wheel and the like, as well as combinations thereof. In an example, activation mechanism 216 can comprise a wheel that can be rotated in one direction to open jaws 212 and rotated in an opposite direction to close jaws 212. For example, the wheel can be rotated to push and/or pull a wire to open and close jaws 212.

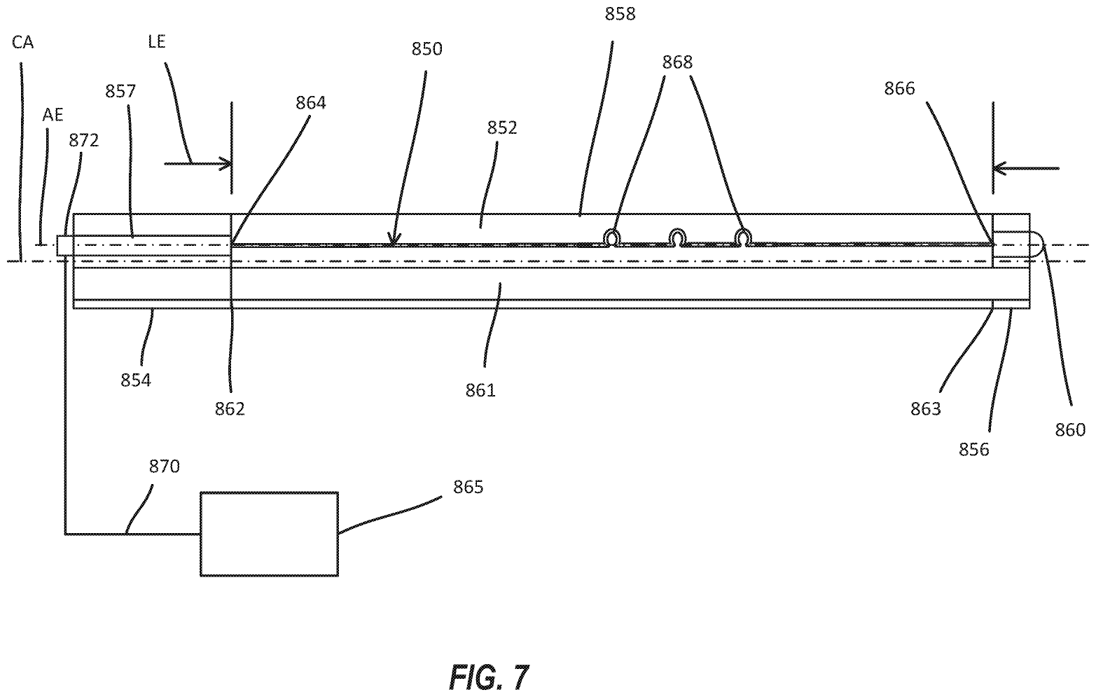

FIG. 5B is a close-up view of a distal end of tissue collection device 204 of FIG. 5A showing translucent tissue separator 210 extending from auxiliary endoscope 230. Endoscope 230 can comprise an example of auxiliary scope 134. Endoscope 230 can comprise shaft 232, working channel 234, passage 236 and lens 238. Field of view 240 can project from lens 238. Endoscope 230 can additionally include lens 239 for the projection of light into field of view 240. In examples, lens 239 can comprise a distal end of optical fiber used to project or emit a laser beam, as described herein. In examples, passage 236 can additionally include a laser fiber in addition to imaging and illuminating wires and components. The devices systems and methods of FIGS. 7-14, including various damage mitigating and preventing devices, can be used with light conducting fibers used within endoscope 230.

Tissue collection device 204 can be configured as a low-profile device so as to be able to be inserted through a small diameter lumen, such as lumen 136 of auxiliary scope 134 of FIG. 4. Additionally, tissue collection device 204 can be configured as a high-capacity tissue collector that can hold a large volume of collected sample tissue to thereby reduce or eliminate the need to repeatedly remove surgical instrument 200 from the auxiliary scope. Furthermore, tissue collection device 204 can be optically enhanced to facilitate user operation of tissue collection device 204 to interact with target tissue. For example, jaws 212A and 212B can be fabricated from translucent material to allow lens 238 to see through jaws 212A and 212B, and teeth 213 can be fabricated of reflective material to reflect light from lens 239 back to lens 238 to allow a user to more clearly delineate where tissue collection device 204 will interact with target tissue of the patient. Jaws 212A and 212B can further be configured to provide magnification of target tissue when viewed through one or both of jaws 212A and 212B. In examples, one or both of jaws 212A and 212B can include one or more convex surfaces of transparent material to provide optical magnification.

Tissue collection device 204 can be fully retracted into working channel 234. Working channel 234 can comprise lumen 136 of FIG. 4. As such, lens 238 can be freely moved by manipulation of shaft 232 to position target tissue within field of view 240. However, when it is desired to extend tissue collection device 204 from working channel 234, tissue collection device 204 can become positioned within field of view 240, thereby inhibiting or preventing lens 238 from capturing images of the target tissue. As discussed with reference to FIGS. 6A-6B, tissue collection device 204 can be configured to allow light to 1) pass through components, portions or all of separator 210, and/or 2) be reflected by components, portions or all of separator 210 to enhance images obtained through lens 238.

FIG. 6A is a schematic illustration of surgical instrument 200 wherein separator 210 comprises forceps in a closed state and extended from endoscope 230 proximate target tissue 254. FIG. 6B is a schematic illustration of surgical instrument 200 with separator 210 in a deployed state with forceps open to engage target tissue 254. FIGS. 6A and 6B are discussed concurrently and the components therein are not necessarily drawn to scale.

As shown in FIG. 6A, tissue collection device 204 can be positioned in an anatomic duct 255 where target tissue 254 is located. Shaft 222 can be used to guide separator 210 through an anatomic duct to target tissue 254. Target tissue 254 can comprise a protrusion, such as a growth of cancerous or pre-cancerous material.

Endoscope 230 can be positioned such that lens 238 faces target tissue 254. As such, target tissue 254 can be within field of view 240 of lens 238. Field of view 240 is illustrated as having a particular viewing angle. However, lens 238 can be configured to have field of view 240 with different angles, up to and including one-hundred-eight degrees. As can be seen in FIG. 6A, tissue collection device 204 extended from shaft 232 to expose jaws 212A and 212B, but to not yet engage target tissue 254. As such, jaws 212A and 212B can thus be located to not completely block field of view 240 from target tissue 254. However, field of view 240 can become obstructed the further tissue collection device 204 becomes extended from working channel 234. For example, the portion of duct 255 from which target tissue 254 extends can become blocked from viewing by lens 238.

FIG. 6B is a side view of tissue collection device 204 with jaws 212 shown in cross-section to show storage space 256 with sample tissue 258. Jaws 212 can be elongated in the radial directions (e.g., up and down with respect to the orientations of FIG. 6B) so as to form a container for the storage of collected matter.

With jaws 212 rotated away from each other at hinge 214, tissue collection device 204 can be moved in the axial direction toward sample tissue 258. Jaws 212 can be rotated toward each other to engage target tissue 254. Tissue collection device 204 can be reciprocated back-and-forth along the axis of shaft 222 to collect sample tissue 258. Teeth 213 can be used to cut, saw, tear or rip portions of target tissue 254 away from the anatomy of the patient. In examples, only one of jaws 212A and 212B can be configured to rotate.

Teeth 213 can be fabricated out of an edge of jaws 212A and 212B. In examples, teeth 213 can comprise extensions of the material of jaws 212A and 212B. In such examples, both teeth 213 and jaws 212A and 212B can be fabricated of a rigid material such as plastic or metal. In examples, jaws 212A and 212B can be fabricated from Gorilla Glass® commercially available from Corning, or other chemically strengthened glass such as alkali-aluminosilicate sheet glass. In examples, jaws 212A and 212B can be fabricated from molded polycarbonate.

In additional examples, teeth 213 and jaws 212A and 212B can be mounted to a frame extending from hinge 214. For example, jaw 212A can comprise a U-shaped, rigid frame having end portions extending from hinge 214 to form a bounded space. Jaw 212A can comprise a bag or bellows of flexible material mounted to the U-shaped, rigid frame to partially enclose the bounded space. Teeth 213 can extend from the U-shaped, rigid frame away from the partially enclosed space. Jaw 212B can be configured similarly with teeth 213 configured to mesh with teeth 213 of jaw 212A. Thus, the flexible material of jaws 212A and 212B can form a full enclose when jaws 212A and 212B are rotate to engage, but can bend to not interfere with teeth 213 engaging target tissue 254.

Teeth 213 can be configured to have one or more orientations. For example, teeth 213 can be angled distally toward target tissue 254, or proximally toward shaft 222. In examples, some of teeth 213 can be angled proximally and some of teeth 213 can be angled distally. In examples, teeth 213 can be oriented in different directions.

As discussed above, components or portions of tissue collection device 204 can be made of optically enhanced materials. In examples, jaws 212A and 212B can be made of translucent or transparent material that can allow light waves to travel therethrough, thereby allowing lens 238 to “see through” jaws 212A and 212B. Transparent materials can allow lens 238 to see native coloring of target tissue 254. Translucent materials can be configured to allow lens 238 to see target tissue 254 in a filtered manner. As such, jaws 212A and 21B can be translucently tinted with different colors to enhance viewing of certain tissue types or mute viewing of other tissue types.

However, in order to maintain control of tissue collection device 204, e.g., to maintain accurate employment of teeth 213, portions of tissue collection device 204 can be opaque, reflective or translucent. In particular, teeth 213 can be made of opaque, reflective or translucent material or can have a coating applied thereto. In examples, teeth 213 can be opaque to be easily viewable by lens 238. In additional examples, teeth 213 can be configured to optically interact with light from lens 239. For example, teeth 213 can have a reflective coating applied thereto, such a coating of grains of reflective particles or titanium oxide. Thus, light from lens 239 can be bounced bac to lens 238. In additional examples, teeth 213 can be fluorescent to light up when engaged by a certain type of light. Thus, light from lens 239 can cause lens 238 to view teeth 213 in a particular wavelength that is more discernable relative to duct 255. In examples, only some of teeth 213 can be reflective or fluorescent.

In view of the foregoing, use of optically enhanced tissue collection devices can facilitate viewing of target tissue 254 through jaws 212A and 212B, viewing of sample tissue 258 within jaws 212A and 212B, and viewing of laceration 260 where sample tissue 258 was removed from target tissue 254. As such, endoscope 230 can be used to view interior tissue layers within laceration 260 and potentially diagnose conditions of the that tissue.

FIG. 7 is a schematic diagram illustrating a length of light-conducting element 850 extending through shaft 852 located between proximal controller 854 and distal end portion 856. Shaft 852 can comprise elongate body 858, lumen 861, proximal end 862 and distal end 863. Shaft 852 can be part of a device for performing a surgical procedure as described herein. As such, shaft 852 can comprise an endoscope, including endoscope 230 of FIGS. 5B-6B. In examples, shaft 852 can be part of a laser lithotripsy device configured to fragment biological stones using laser energy transmitted through light-conducting element 850. Light-conducting element 850 can be a multi-purpose light transmitter configured to conduct laser energy for fragmentation and light energy for illumination and energizing dye as described herein. As such, shaft 852 can comprise a single-use, multi-function scope that enables manufacture of a single device with features to enable multiple different functionalities.

Controller 854 can comprise a device located at proximal end 862 of shaft 852 and can be configured to operate components of shaft 852 and components attached thereto. As such, controller 854 can include various control knobs, buttons and the like for operating steering capabilities of shaft 852. Controller 854 can comprise socket 857 for receiving light-conducting element 850. Socket 857 can be configured to connect light-conducting element 850 to laser module 865 and a light source, such as one on control unit 16 (FIG. 1). Controller 854 can include various control knobs, buttons and the like for operating laser module 865 and the light source. Controller 854 can be configured similarly as any of the controllers described herein, such as controller 206 of FIG. 5A.

Distal end portion 856 can comprise a cap located at distal end portion 856 of shaft 852 to seal-off lumen 861 from the environment of shaft 852. Distal end portion 856 can comprise a platform for mounting other components, such as lens 860 that discharges laser energy from light-conducting element 850. Lens 860 can be connected to light-conducting element 850. In additional examples, lens 860 can be omitted such that laser energy can be discharged directly from light-conducting element 850. Distal end portion 856 can be configured similarly as other components described herein, such as camera module 50 of FIG. 3B.

Shaft 852 is illustrated as including light-conducting element 850 and lumen 861, but as referenced above, can include other elements and components such as cables, tubes and the like to facilitate other capabilities, such as imaging and irrigation.

Light-conducting element 850 can be used to conduct laser light from proximal controller 854 to distal end portion 856. Laser module 865 can be connected to socket 857 of controller 854 via cable 870 and connector 872. Light-conducting element 850 can provide a connection between laser module 865 and lens 860. As such, laser energy from laser module 865 can be transmitted to distal end portion 856 to provide energy for fragmenting stones and the like. In examples, laser module 865 can be configured to generate laser energy to fragment stones as is described in previously mentioned U.S. Pat. No. 10,646,276 to Fan et al. and U.S. Pat. No. 9,259,231 to Navve et al. In examples, laser module 865 can comprise a thulium fiber laser module. In examples, laser module 865 can comprise a Soltive™ SuperPulsed Laser System from Olympus®.

In examples, light-conducting element 850 can comprise a fiber or filament capable of transmitting light and in particular laser light. Light-conducting element 850 can comprise a medium for transmitting light from laser module 865 to lens 860. In examples, light-conducting element 850 can be made from silica, fluorozirconate, fluoroaluminate, chalcogenide glasses, and crystalline materials such as sapphire. Light-conducting element 850 can comprise a material suitable for transmitting waves of electromagnetic radiation at various wavelengths. Cable 870 can comprise an extension of light-conducting element 850 and can be fabricated from the same material as light-conducting element 850. In examples, light-conducting element 850 and cable 870 can comprise fiber optic cables. In examples, the fiber optic cables can comprise glass and plastic fibers jacketed with one or more protective and reflective coatings. Lens 860 can be located at or near the distal end of light-conducting element 850. Lens 860 can be coupled to light-conducting element 850 by any suitable means. In examples, lens 860 can comprise any suitable light emitter for collecting and focusing light waves from light-conducting element 850. Lens 860 can comprise a glass or plastic body of transparent material. However, in additional examples, a separate light emitter is not used and light-conducting element 850 can comprise an end-emitting fiber such that the distal or terminal end of light-conducting element 850 can comprise a light emitter. In examples, light-conducting element 850 can have a circular cross-sectional area having a diameter in the range of approximately 250 microns (μm/1×10⁻⁶ meter) to 500 microns (μm/1×10⁻⁶ meter). Additionally, in examples using thulium fiber laser modules, light-conducting element 850 can have a circular cross-sectional diameter in the range of approximately 50 microns to 150 microns.

In various examples, light-conducting element 850 can extend between controller 854 and end portion 856. For example, light-conducting element 850 can be attached to controller 854 at fixed point 864 and attached to end portion 856 at fixed point 866. Fixed points 864 and 866 do not necessarily correspond to the diametric ends of light-conducting element 850 such that ends of light-conducting element 850 can extend into controller 854 and end portion 856, respectively. Fixed points 864 and 866 can, therefore, represent locations within shaft 852 where light-conducting element 850 is connected to other components and the like such that the length of light-conducting element 850 between fixed points 864 and 866 is continuous and unfixed or unpinned.

When shaft 852 is in a straight position, shaft 852 can have length LE extending straight along central axis CA. Light-conducting element 850 can extend along axis AE. As shaft 852 bends, light-conducting element 850 can become subject to loading, such as strain from being stretched or other bending stresses. In particular, if axis AE is positioned offset from center axis CA of shaft 852, bending of shaft 852 can cause tension in light-conducting element 850, particularly when bent in the direction opposite the direction that axis AE is offset from center axis CA. Furthermore, when shaft 852 is bent at a tight angle, such as a ninety-degree angle or thereabouts, the stress can be exacerbated.

With the present disclosure, light-conducting element (light conductor) 850 can include slack 868 between fixed points 864 and 866 to allow light-conducting element 850 to bend with shaft 852 without being subject to loading that produces undesirable stress or strain within light-conducting element 850. As such, light-conducting element 850 can be longer than shaft length LE between fixed points 864 and 866. Slack 868 can, therefore, take up the excess length of light-conducting element 850 beyond length LE. In examples, “slack” can comprise extra length of a light conductor to provide strain relief. As such, “slack” as used herein can be greater than sagging or drooping of a light conductor that is intended to extend along a straight line, but that sags or droops due to gravity. For example, space within a typical medical scope is constrained such that a light conductor would not be permitted to sag to a level to provide strain relief. However, slack 868 contemplated by the present disclosure can comprise formations, such as loops, coils, undulations or bunching of light-conducting element 850 or other formations of light-conducting element 850 that can allow for the shaping of a light-conducting element 850 that is longer than length LE. Slack 868 can thus provide a strain relief feature to the potential stress and strain that can be introduced due to bending, such as that discussed above. In additional examples, it is not necessary for light-conducting element 850 to be pinned at proximal and distal portions within the scope for the slack to provide strain relief.

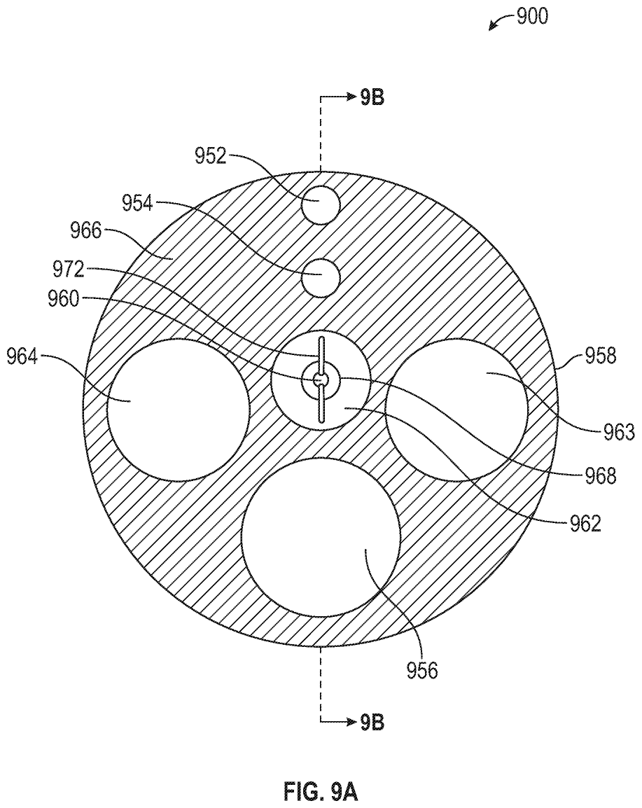

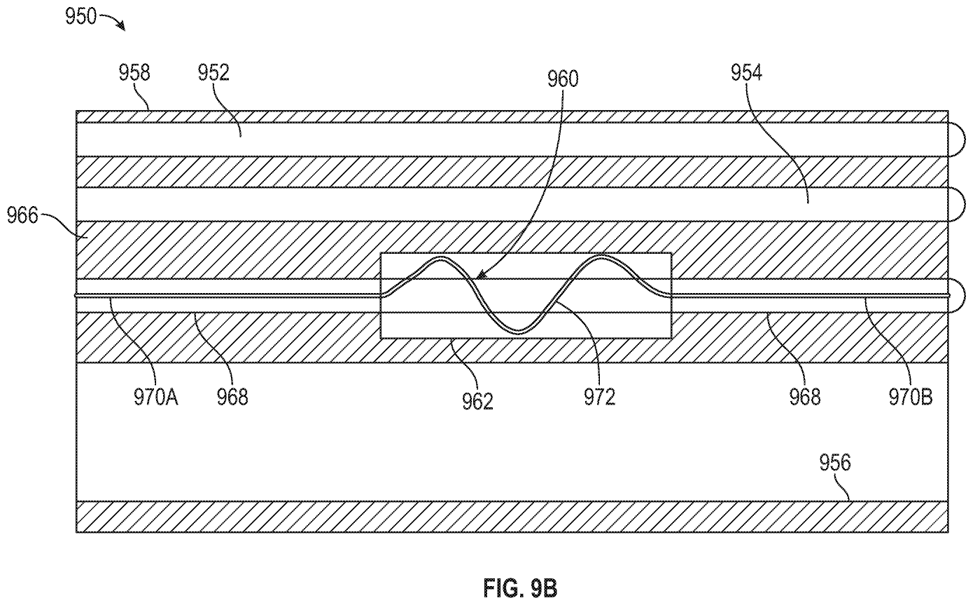

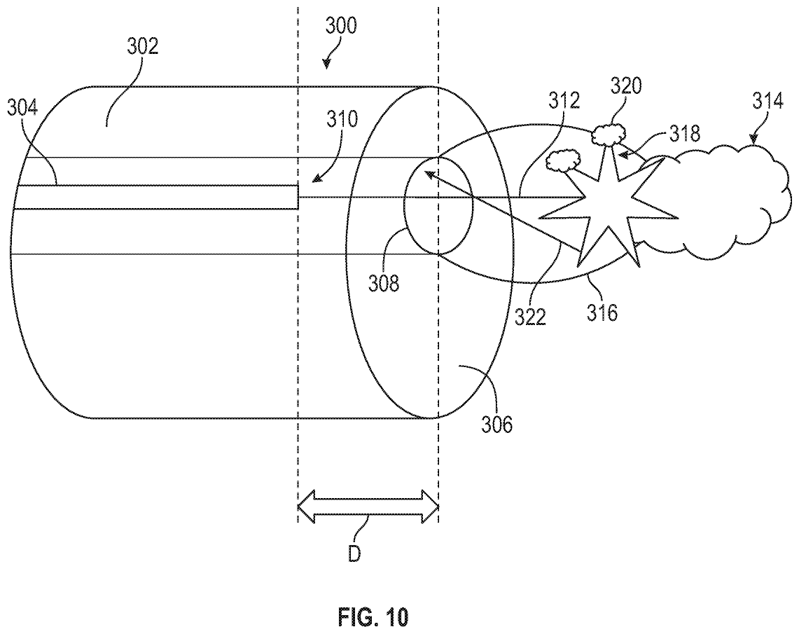

Slack 868 is illustrated as being proximate distal end portion 856, but can be located anywhere along the length of light-conducting element 850 and shaft 852 in a uniform or non-uniform distribution. In examples, slack 868 can be located at the axial position along shaft 852 where the most severe bending is expected to occur, such as where a scope is expected to turn between a duodenum and a common bile duct. In examples, slack 868 can be located approximately 30 millimeters from the distal end of shaft 852 or within the distal most 25% of shaft 852. Slack 868 can be freely disposed within shaft 852 alongside, about or around other components of shaft 852, as shown in FIGS. 8A and 8B, or can be contained within a lumen for light-conducting element 850 having a pocket for slack 868, as shown in FIGS. 9A and 9B. FIGS. 8A-9B illustrate two examples of devices of the present disclosure that can be modified, adapted or combined to accommodate slack 868 in various scope shaft designs. Furthermore, the examples of FIGS. 8A-9B can be combined with other devices and features discloses herein, particularly those damage preventing and mitigating devices discussed with reference to FIGS. 10-13D.

FIG. 8A is a schematic cross-sectional view of scope shaft 900 comprising light conductor 902, imaging cable 904 and working channel 906 extending through tubular sheath 908 including loosely coiled light-conducting element 910. FIG. 8B is a schematic side view of scope shaft 900 of FIG. 8A showing loosely coiled light-conducting element 910 wrapped around light conductor 902 and imaging cable 904. FIG. 8A also shows additional passages 912 and 914 extending through tubular sheath 908. Scope shaft 900 can optionally include lenses 915A, 915B and 915C. FIGS. 8A and 8B are discussed concurrently.

FIGS. 8A and 8B illustrate light-conducting element 910 incorporated into an end-viewing endoscope, such as a gastroscope, colonoscope, or cholangioscope. However, light-conducting element 910 can be incorporated in other types of scopes, such as side-viewing endoscopes including duodenoscopes. In examples, light-conducting element 910 can be incorporated into endoscope 230 of FIGS. 5A and 5B.

Light conductor 902, imaging cable 904, working channel 906 and additional passages 912 and 914 can be positioned within lumen 918 of tubular sheath 908. Some of space 916 within lumen 918 can be unoccupied. Light conductor 902, imaging cable 904, working channel 906 and additional passages 912 and 914 are not necessarily drawn to scale relative to lumen 918 and each other. However, space 916 between light conductor 902, imaging cable 904, working channel 906 and additional passages 912 and 914 can provide space for light-conducting element 910. As such, rather than providing light-conducting element 910 within a sheath or tube that extends straight through sheath 908, light-conducting element 910 can extend, partially or wholly, within space 916 so as to be able to be non-linear in shape. Space 916 can provide room for light-conducting element 910 to accumulate a length of material greater than what is necessary to span the length of sheath 908. As such, light-conducting element 910 can accumulate slack suitable to allow light-conducting element 910 to bent without or with minimal bending stresses. Thus, space 916 can provide an operating envelope for light-conducting element 910 to be shaped, bent or curved to include slack, such as coiling or undulations, to allow for bending of light-conducting element 910 with little or no stress.

Light-conducting element 910 can comprise straight sections 920A and 920B with loops 922 located therebetween to form coil 924. In examples, light-conducting element 910 can be matched to curvature of sheath 908 so that loops 922 abut sheath 908. Sheath 908 can thereby provide support to light-conducting element 910.

In the illustrated example of FIGS. 8A and 8B, light-conducting element 910 is wrapped around light conductor 902, imaging cable 904, working channel 906 and additional passages 912 and 914 to form coil 924. However, light-conducting element 910 can be wrapped around fewer of those elements. In additional embodiments, light-conducting element 910 can be coiled within space 916 without being wrapped around any other component or element. In examples, loops 922 of coil 924 can be matched to curvature of sheath 908.

Coil 924 can have spacing length L1 (FIG. 8B) between individual loops and each individual loop can generally have a radius R1 (FIG. 8A). Length L1 and radius R1 can be adjusted based on design needs to fit an appropriate length of light-conducting element 910 within space 916. In examples, the longer length L1 is, the larger radius R1 becomes, which can facilitate providing excess slack for light conductor 902 without exceeding recommended or estimated bending limits of the material of light conductor 902. However, Length L1 and radius R1 can be set based on the material properties of light-conducting element 910 to prevent or minimize formation of stress within light-conducting element 910. As discussed, light-conducting element 910 can have a diameter of about 250 microns to about 500 microns in various examples or be as small as 100 microns. Different light-conducting elements can have different minimum bend radiuses determined by a manufacturer. However, a rule for general guidance is that the minimum bend radius can be equal to approximately ten times the outer diameter of the light-conducting element to avoid stressing light-conducting element 910. It is, however, possible to have smaller minimum bend radiuses, particularly if the light-conducting fiber is not intended to be reused as in the case of disposable, single-use scopes. Thus, per the general rule, for a light-conducting element having an outer diameter of 250 microns, the minimum bend radius can be set to 2.5 millimeters (mm), and for a light-conducting element having an outer diameter of 500 microns, the minimum bend radius can be set to 5.0 mm. For a light-conducting element having an outer diameter of 100 microns for use with a thulium fiber laser module, the minimum bend radius can be set to 1.0 millimeters (mm) per the general rule. As mentioned, the minimum bend radiuses of these listed examples can be smaller if the light-conducting element 910 is used in a disposable, single-use device.

In examples, the outer diameter of sheath 908 can be approximately 3.4 mm and radius R1 can be set to be approximately 1.0 mm to approximately 3.3 mm.

In examples, light-conducting element 910 can be configured to be straight at rest and then subjected to bending forces to include the shape of coil 924 as disposed inside of sheath 908. In examples, light-conducting element 910 can be formed to include the shape of coil 924 when at rest. For example, light-conducting element 910 can be wound to include coil 924 and then heat treated to provide thermal stress relief. In examples, light-conducting element 910 can be heat treated as is known in the art. See Lezzi, Peter & Tomozawa, M. (2014). Strength increase of silica glass fibers by surface stress relaxation—A new mechanical strengthening method. American Ceramic Society Bulletin. 93. 36-39.

FIG. 9A is a schematic cross-sectional view of scope shaft 950 comprising additional passages 952 and 954 and working channel 956 extending through shaft body 958 including loosely coiled light-conducting element 960. FIG. 9B is a schematic side view of scope shaft 950 of FIG. 9A showing loosely coiled light-conducting element 960 disposed within slack chamber 962. FIG. 9B additionally shows working channel 956 and additional passages 952 and 954 extending through shaft body 958. FIGS. 9A and 9B are discussed concurrently.

FIGS. 9A and 9B illustrate light-conducting element 910 incorporated into an end-viewing endoscope, such as a gastroscope, colonoscope, or cholangioscope. However, light-conducting element 910 can be incorporated in other types of scopes, such as side-viewing endoscopes including duodenoscopes.

Additional passages 952 and 954, working channel 956 and lumens 963 and 964 can be formed out of the material of scope shaft 950. As such, scope shaft 950 can be fabricated as a solid, elongate body having lumens extending therethrough. Some of places 966 within scope shaft 950 can be unoccupied by a lumen and thus can provide the location for lumen 968 for light-conducting element 960. Additional passages 952 and 954, working channel 956 and lumens 963 and 964 are not necessarily drawn to scale relative to lumen 968 and each other. However, places 966 between additional passage 952, additional passage 954, working channel 956 and lumens 963 and 964 can provide space for light-conducting element 960. Slack chamber 962 can be located along lumen 968 to allow light-conducting element 960 to be non-linear in shape. Slack chamber 962 can provide room for light-conducting element 960 to accumulate a length of material greater than what is necessary to span the length of scope shaft 950. As such, light-conducting element 960 can accumulate slack suitable to allow light-conducting element 960 to bent without or with minimal bending stresses. Thus, slack chamber 962 can provide an operating envelope for light-conducting element 960 to be shaped, bent or curved to include slack, such as coiling or undulations, to allow for bending of light-conducting element 960 with little or no stress.

Light-conducting element 960 can comprise straight sections 970A and 970B with undulations 972 located therebetween. In additional examples, slack in light-conducting element 960 can be provided by other formations than undulations 972, such as bunching or coiling. In examples, light-conducting element 960 can include coiling that matches curvature of slack chamber 962 so that slack chamber 962 can thereby provide support to light-conducting element 960.

Undulations 972 can be shaped and formed as described herein to reduce or eliminate bending stresses, such as by including a minimum radius of curvature for light-conducting element 960. Undulations 972 can additionally be temporarily of permanently formed into light-conducting element 960 as described herein, such as by using thermal stress relief techniques.

In the illustrated example, slack chamber 962 is shown as comprising a cylindrical shape extending along the center axis of scope shaft 950 over an axial sub-segment of shaft 950. However, slack chamber 962 and lumen 968 can have other locations offset from the center axis of scope shaft 950. Additionally, slack chamber 962 can have other shapes, such as rectangular, square or arcuate. In an example, an arcuate (curved in the circumferential direction) slot having a radial thickness and an axial length can be positioned between the center axis of scope shaft 950 and the exterior of scope shaft 950. In another example, slack chamber 962 can comprise a quarter section or half section of scope shaft 950 extending from the center axis of scope shaft 950 to a radial extend within scope shaft 950.

In examples, slack chamber 962 can comprise substantially all of the interior portions of scope shaft 950 such that additional passages 952 and 954, working channel 956 and lumens 963 and 964 additionally pass through slack chamber 962. Thus, a hybrid of the example of FIGS. 8A and 8B and the example of FIGS. 9A and 9B can be produced wherein light-conducting element 960 is disposed within lumen 968 along straight portions, but can be located within the equivalent of space 916 (FIG. 8A) to allow light-conducting element 960 to be expanded with larger radius of curvatures than slack chamber 962 of FIGS. 9A and 9B. Shaft 950 can thus be solid where light-conducting element 960 is straight and can be the equivalent of a sheath where light-conducting element is loosely coiled or bunched to provide stress relief capabilities.

The present disclosure provides a light-conducting element that can be incorporated into elongate surgical instruments, such as endoscopes, that are subject to bending stresses to allow the light-conducting element to be commensurately bent without being subject to bending stresses that have the potential to damage the light-conducting element. The light-conducting elements of the present disclosure can include slack that results from the light-conducting element being longer than a shaft of the elongate surgical instrument so that when the light-conducting element is bent, the slack is taken up rather than the light-conducting element being subject to bending stresses or tension. The slack can be provided in various formations of the light-conducting elements, such as coils, bunches, undulations and the like. The slack can be positioned within locations of the elongate surgical instrument that is unoccupied by other components. The slack can be located anywhere along the length of the light-conducting element or anywhere along the length of the elongate surgical instrument. The light-conducting element can be suitable for delivering fragmentation energy or illumination/dye-energizing light, such as can be advantageously incorporated into multi-function, single-use devices. The light-conducting element can have a small diameter to facilitate bending and minimize space impact with elongate surgical instruments. The light-conducting element can be adequately sized to deliver laser fragmentation energy for various biological stones, particular bile duct stones.

FIG. 10 is a schematic illustration of scope 300 comprising scope shaft 302 having integrated fiber 304 that is recessed from distal end surface 306 of scope shaft 302. Fiber 304 can be located in lumen 308. Distal end 310 of fiber 304 can be recessed distance D from distal end surface 306.

Lumen 308 can comprise an extension of a lumen holding fiber 304. Lumen 308 can be fabricated from the material of shaft 302. In examples, shaft 302 can be made of a plastic or polymer. In examples, lumen 308 can be fabricated as a separate component attached to shaft 302, such as on an end cap that fits over shaft 302 and that incorporates lumen 308 across distance D to align with fiber 304. The size, e.g., diameter and length, of lumen 308 relative to fiber 304 can be selected to achieve the desired protective and energy mitigating effects as described herein. The diameter of fiber 304 is exaggerated in FIG. 10 relative to the diameter of lumen 308 for illustrative purposes. The diameter of lumen 308 across distance D can be greater or smaller than the diameter of lumen 308 in shaft 302 where fiber 304 is located. Distance D can be fixed due to, for example, the pinning of fiber 304 according to the examples described herein. Distance D can be set at a length to provide for development of shockwave 316 suitable for fragmenting matter 314. The fixed arrangement of fiber 304 can also provide user of scope 300 a consistent and repeatable experience.

Fiber 304 can emit laser beam 312 to impact biological matter 314, which can comprise a kidney stone or a gallstone. Laser beam 312 can enter fluid surrounding scope shaft 302 to generate shockwave 316. In examples, the diameter of lumen 308 can be sized to allow fluid into lumen 308 to facilitate formation of shockwave 316. Shockwave 316 can cause release of energy 318 from matter 314 resulting in fragments 320 being formed. Matter 314 can cause laser energy 322 to be deflected back toward scope 300. Although only one instance of deflected laser energy is illustrated, laser energy can be deflected back toward scope 300 in a plurality of different directions based on, for example, the differing surface angles of matter 314 and other factors. Likewise, various sized fragments 320 can explode from matter 314 in various different directions.

Lumen 308 can comprise a protection device to potential damage-causing occurrences of laser energy, shockwaves, stone fragment impacts and thermal damage. First, material of scope shaft 302 extending beyond fiber 304 to form distal end surface 306 can prevent reflected laser energy 322 from touching distal end 310 of fiber 304. Similarly, distal end surface 306 can deflect fragments 320 that are directed toward scope 300, thereby preventing damage from kinetic and thermal energy of fragments 320. Second, material of scope shaft 302 extending beyond fiber 304 to form lumen 308 can prevent reflected laser energy 322 from reaching distal end 310 of fiber 304 in a direct matter. For example, laser energy 322 entering lumen 308 at an angle can be incident on a sidewall of lumen 308. Thus, laser energy 322 can bounce within lumen 308 before reaching distal end 310 of fiber 304, thereby dissipating the magnitude of laser energy 322 to levels potentially below levels that can cause damage. In examples, lumen 308 can be coated with a material to mitigate laser energy 322, such as an anti-reflective coating. Similarly, lumen 308 can prevent large fragments 320 from reaching fiber 304. Smaller fragments 320 that enter lumen 308 can have less kinetic energy and can retain less heat, thereby mitigating the risks to fiber 304.

The diameter and length of distance D can be varied. For example, the diameter of lumen 308 can be decreased to limit the amount of laser energy 322 that can enter lumen 308 and the length of distance D can be increased to allow more bouncing or deflecting of laser energy 322.

Thus, lumen 308 can limit the instances of full power laser energy 322 that are reflected back to fiber 304 to only those that can make it straight through lumen 308 without hitting a sidewall of lumen 308. As such, the number of instances of reflected laser energy 322 can be greatly reduced compared to configurations where distal end 310 extends through lumen 308 to distal end surface 306.

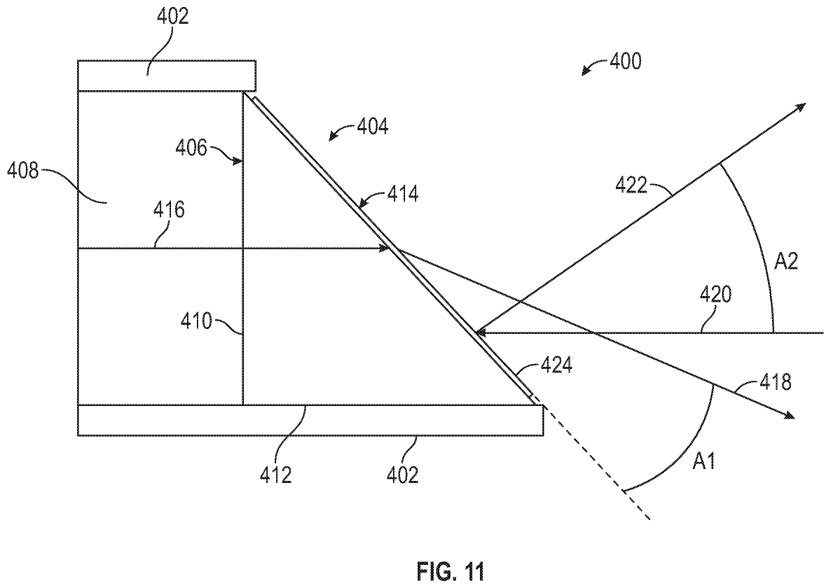

FIG. 11 is a schematic illustration of scope 400 comprising scope shaft 402 having prism 404 disposed at distal end surface 406 at integrated fiber 408. Prism 404 can comprise base surface 410, side surface 412 and deflector surface 414. Integrated fiber 408 can emit laser beam 416, which can be bent by deflector surface 414 at angle A1 to produce emitted laser beam 418. Reflected laser light 420 can be deflected by deflector surface 414 at angle A2 to produce deflected laser beam 422.

Prism 404 can be configured as a body of material that optically allows laser beam 416 to pass therethrough without, or with minimal, loss of intensity, but that prevents stray or reflected laser light from passing through in the opposite direction. In examples, prism 404 can be made of glass, acrylic or fluorite.

Prism 404 can be configured to alter the trajectory of laser beam 416. Because laser beam 416 comprises a single wavelength of light, all of laser beam 416 can be bent by prism 404 in a single direction indicated by deflected laser beam 422. The angles between surfaces 410, 412 and 414 can be adjusted along with the orientation of prism 404 relative to central axis of scope shaft 402 to control the direction of emitted laser beam 418. In the illustrated example, prism 404 can be configured as a right-angle prism where the angle between surface 410 and surface 412 is approximately ninety degrees, and surface 410 can be oriented approximately perpendicular to the central axis of scope shaft 402. Likewise, the position and geometry of prism 404 can be selected based on the medium into which emitted laser beam 418 is released. For example, water can have a refraction index of 1. 3, while air has a refraction index of 1. In examples, the geometry and orientation of prism 404 along with selection of the laser transmitting medium can be selected to minimize angle A1 to facilitate aiming of emitted laser beam 418 close to the axis of scope shaft 402. Because stones are typically placed close to the end of a laser lithotripsy device for fragmentation, typically around 1 mm, the effects of angle A1 can be minimally perceived.

Deflector surface 414 can include coating 424. In examples, coating 424 can comprise a mirror coating configured to allow light to pass therethrough from one side and deflect or reflect light from the opposite side. In examples, coating 424 can comprise a half-silvered surface coating. Thus, laser beam 416 can pass through coating 424, but reflected laser light 420 is prevented from entering prism 404 and is reflected back away from fiber 408. In examples, angle A2 can be set at any angle to deflect laser energy away from scope 400. However, as mentioned, because scope 400 is configured to operate in close proximity to stones, angle A2 can be selected to direct laser energy back to stones to facilitate the fragmentation process.

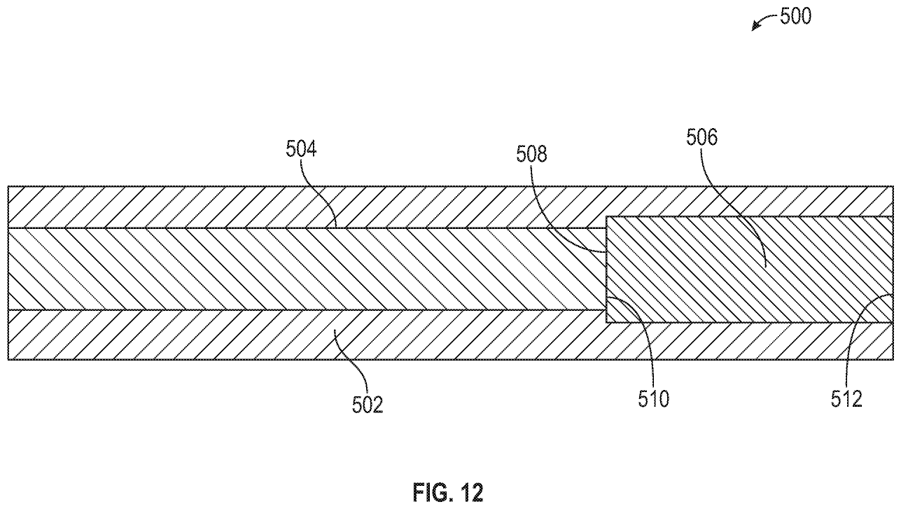

FIG. 12 is a schematic illustration of scope 500 comprising scope shaft 502 having light-conducting fiber 504 and shield 506 placed at distal end 508. Shield 506 can comprise proximal face 510 and distal face 512.

Shield 506 can comprise a body of material that is more robust, more durable and more readily able to withstand inflicted damage than the material of fiber 504. In examples, fiber 504 can be made of silica, quartz and coated silica or quartz. In examples, shield 506 can be made sapphire. In additional examples, shield 506 can be made of diamond or moissanite. Shield 506 can be made of other light-conducting, hard, impact-resistant and heat-resistant material.

Shield 506 can be configured to not interfere with a laser beam leaving fiber 504. As such, a laser beam leaving fiber 504 at a trajectory, e.g., parallel to the axis of fiber 504 at distal end 508, can likewise leave shield 506 at the same trajectory. Likewise, the laser beam can have the same intensity entering shield 506 as when leaving shield 506.

Shield 506 can be configured to receive reflected laser light and fragments of broken stones. Shield 506 can receive reflected laser light an endure the initial blow and then dissipate the laser energy before it reaches fiber 504. Likewise, shield 506 can absorb the impact of any stone fragments as well as any heat input from the stone fragments.

In the illustrated example, shield 506 can comprise a cylindrical body having an outer diameter slightly larger than the diameter of light-conducting fiber 504. However, shield 506 can have any cross-sectional geometry adequate to cover distal end 508 of fiber 504 including having the same diameter. Shield 506 can have any suitable axial length. For example, shield 506 can have any axial length suitable to dissipate or withstand laser energy as described herein. In examples, shield 506 can have a length commensurate with distance D of FIG. 10.

Shield 506 can be in optical communication with fiber 504. Shield 506 can be placed to abut fiber 504. In examples, shield 506 can be attached to fiber 504, such as with a light-transmitting glue or adhesive. In examples, shield 506 can be uncoupled and spaced apart from the distal end of fiber 504.

In examples, shield 506 can be configured to be a replaceable component, either by the user or at a remanufacturing facility. Shield 506 can be held in place with a removable cap that holds shield 506 at the end of shaft 502. In examples, shaft 502 can include a resilient socket into which shield 506 can be inserted for retention and that can be stretched for removal or insertion of shield 506. Thus, shield 506 can comprise a consumable component that can be degraded by laser energy, stone fragments and heat energy, and then replaced with a new shield. Shield 506 can thereby be made of a minimal amount of material to reduce costs.

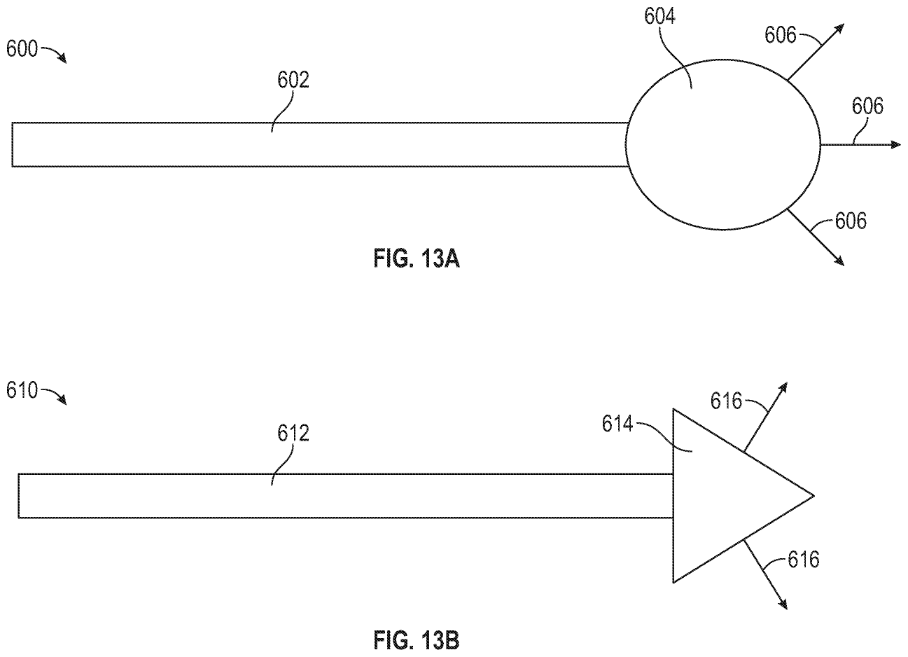

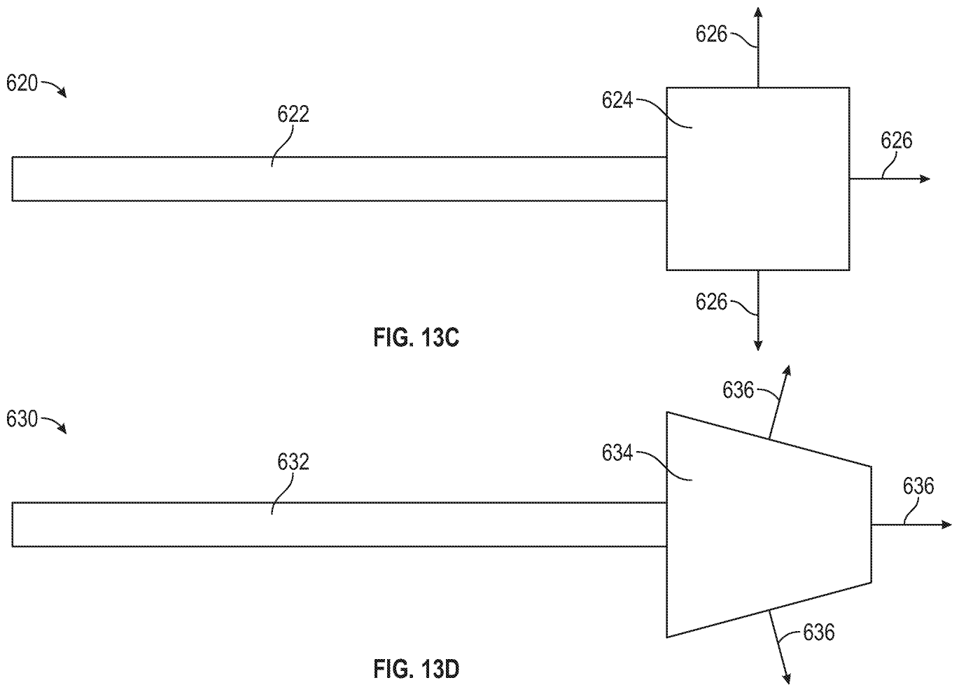

FIG. 13A is a schematic illustration of distal end 600 of light-conducting fiber 602 having bulbous tip 604. Light conducting fiber 602 can be used as any of the light-conducting fibers, light-emitting fibers and the like as described herein. Bulbous tip 604 can be configured to disperse, scatter or spread out a laser beam travelling through light-conducting fiber 602. In the illustrated example of FIG. 13A, bulbous tip 604 can comprise an outer surface having one or more curve geometries that produces surfaces oblique and non-parallel to the central axis of fiber 602. In examples, bulbous tip 604 can be round, oval, teardrop-shaped, capsule-shaped, elliptical and the like. The curvature of the outer surface of bulbous tip 604 can produce scattered laser beams 606. Scattered laser beams 606 can be normal to the surface of bulbous tip 604. Scattered laser beams 606 can influence the shape of the bubble in the liquid into which a laser beam is emitted to control the shockwave. Scattered laser beams 606 can present a reduced laser power density to biological matter, such as a stone. The reduced power density can heat the stone over a larger area, but can still fragment the stone without causing over-heating of fragments that can bounce back to fiber 602 and cause heat damage.

Bulbous tip 604 can be integral with fiber 602. In examples, bulbous tip 604 can be fabricated from the same material as fiber 602. Thus, in examples, bulbous tip 604 can comprise an extension of fiber 602. Bulbous tip 604 can be formed by heating the material of the distal tip of fiber 504 and then molding the molten material to the shape of bulbous tip 604. In examples, fiber 602 and bulbous tip 604 can be made of silica and combinations of silica and sapphire. In additional examples, bulbous tip 604 can comprise a separate component positioned distally of fiber 602 to optically engage with laser light emitting from fiber 602. For example, bulbous tip 604 can comprise an enlarge lens configured to bend a laser beam in different directions.

FIG. 13B is a schematic illustration of distal 610 end of light-conducting fiber 612 having triangular-shaped tip 614. The angled outer surfaces of triangular-shaped tip 614 can produce scattered laser beams 616. Scattered laser beams 616 can be normal to the surface of triangular-shaped tip 614.

FIG. 13C is a schematic illustration of distal end 620 of light-conducting fiber 622 having a square-shaped tip 624. The angled outer surfaces of square-shaped tip 624 can produce scattered laser beams 626. Scattered laser beams 626 can be normal to the surface of square-shaped tip 614.

FIG. 13D is a schematic illustration of distal end 630 of light-conducting fiber 632 having trapezoid-shaped tip 634. The angled outer surfaces of trapezoid-shaped tip 634 can produce scattered laser beams 636. Scattered laser beams 636 can be normal to the surface of trapezoid-shaped tip 634.

Triangular-shaped tip 614, square-shaped tip 624 and trapezoid-shaped tip 634 can be configured similar to bulbous tip 604, but with different geometry. Tips of other shapes can also be used. Bulbous tip 604, triangular-shaped tip 614, square-shaped tip 624 and trapezoid-shaped tip 634 can be configured to disperse, scatter or spread out a laser beam in differing directions and densities.

In examples, Bulbous tip 604, triangular-shaped tip 614, square-shaped tip 624 and trapezoid-shaped tip 634 can be configured as inserts for the distal tip of an endoscope. The inserts can be configured as optical devices of light transmitting properties that can be attached to a lumen or socket at the end of a light conductor. Thus, a surgeon can select a tip to produce a desired laser density and pattern that can treat stones of different sized or different amounts of stones at the same time.

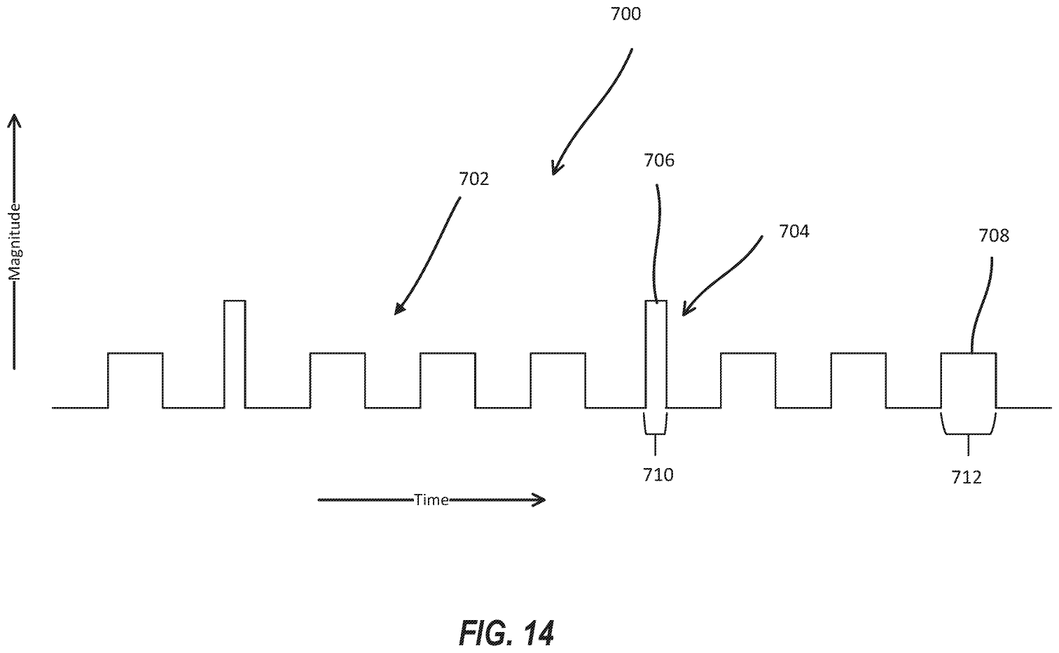

FIG. 14 is a schematic illustration of waveform 700 for generating a laser beam of the present disclosure. In another example of the present disclosure, different types of laser beam pulses can be passed through any of the fibers disclosed herein to clean dirt, dust, debris and the like that may become deposited on the fiber or in proximity to the fiber. Waveform 700 can comprise fragmenting pulses 702 and cleaning pulses 704. Fragmenting pulses 702 can comprise laser pulses suitable for fragmenting biological matter, such as kidney stones and gallstones. Cleaning pulses 704 can comprise laser pulses configured to evaporate or vaporize stone dust particles or break up small stone fragments that have settled onto a laser-emitting scope as described herein. Cleaning pulses 704 can additionally dislodge or displace solid matter that has settled onto the scope.

In examples, fragmenting pulses 702 can typically last for over a second to a few seconds and can have a magnitude of about 20 Watts. Cleaning pulses 704 can comprise bursts that are much shorter in length and much greater in magnitude than fragmenting pulses 702. For example, magnitude 706 of cleaning pulses 704 can be much larger than magnitude 708 of fragmenting pulses 702. Also, duration 710 of cleaning pulses 704 can be much shorter than duration 712 of fragmenting pulses 702. In examples, cleaning pulses 704 can typically last for about one second or less and can have a magnitude of about 30 Watts to about 40 Watts. In examples, cleaning pulses 704 can have a power output about 20% to about 30% higher than the power for fragmenting pulses 702. The short duration of cleaning pulses 704 can help avoid damaging of tissue.

In examples, control unit 16 (FIGS. 1 and 2) can be configured to automatically perform cleaning pulses 704 without user intervention. In examples, control unit 16 can initiate a cleaning pulse at regular intervals, such as time intervals or after a number of fragmenting pulses 702. For example, control unit 16 can initiate a cleaning pulse 704 every five minutes or after every five fragmenting pulses 702. In examples, control unit 16 can sense a drop in power during fragmenting pulses 702. As such, control unit 16 or any of the devices described herein can include a power sensor or circuitry configured to sense power output of a laser generator.

### Various Notes & Examples

Example 1 is a device for performing a surgical procedure, the device comprising: a shaft extending from a proximal portion to a distal portion; a light conductor extending at least partially through the shaft to be exposed at the distal portion; and a damage mitigator positioned to receive light from the light conductor to discharge the light from the device.

In Example 2, the subject matter of Example 1 optionally includes wherein the damage mitigator is configured to protect the light conductor from incoming energy comprising at least one of laser energy, thermal energy and kinetic energy.

In Example 3, the subject matter of any one or more of Examples 1-2 optionally include wherein: the light conductor is pinned at a proximal location and at a distal location; the shaft spans a first length between the proximal location and the distal location; and the light conductor has a second length between the proximal location and the distal location that is greater than the first length to produce slack in the light conductor.

In Example 4, the subject matter of any one or more of Examples 1-3 optionally include wherein: the light conductor includes a distal end; and the shaft comprises a distal end face including an opening to receive the distal end of the light conductor.

In Example 5, the subject matter of Example 4 optionally includes wherein the damage mitigator comprises a lumen extending into the distal end face to receive the distal end of the light conductor, wherein the distal end of the light conductor is positioned a distance from the distal end face of the shaft within the lumen.

In Example 6, the subject matter of Example 5 optionally includes wherein the light conductor has a first diameter and the lumen has a second diameter, wherein the second diameter is different than the first diameter.

In Example 7, the subject matter of Example 6 optionally includes wherein the distance is fixed.

In Example 8, the subject matter of any one or more of Examples 1-7 optionally include wherein the damage mitigator comprises an optical device comprising: a first side facing the light conductor; and a second side facing away from the light conductor; wherein the optical device is configured to allow light from the light conductor to pass through; and wherein the optical device is configured to reflect light at the second side.

In Example 9, the subject matter of Example 8 optionally includes wherein the optical device comprises a prism.

In Example 10, the subject matter of any one or more of Examples 8-9 optionally include wherein the second side includes a mirror coating.

In Example 11, the subject matter of any one or more of Examples 1-10 optionally include wherein the damage mitigator comprises a shield, the shield being formed of a material that is light transmitting and harder than the material of the light conductor.

In Example 12, the subject matter of Example 11 optionally includes wherein the shield comprises a sapphire body and the light conductor comprises silica or quartz.

In Example 13, the subject matter of any one or more of Examples 11-12 optionally include wherein the shield has a first diameter and the light conductor has a second diameter, wherein the second diameter is larger than the first diameter.

In Example 14, the subject matter of any one or more of Examples 11-13 optionally include wherein the shield comprises an anti-reflective coating.

In Example 15, the subject matter of any one or more of Examples 11-14 optionally include wherein the shield is uncoupled from the light conductor.

In Example 16, the subject matter of any one or more of Examples 1-15 optionally include wherein the damage mitigator comprises an enlarged lens located at a distal end of the light conductor, the enlarged lens including at least one surface that is non-parallel to a central axis of the light conductor.

In Example 17, the subject matter of Example 16 optionally includes wherein the enlarged lens comprises an enlarged portion of a distal end of the light conductor.

In Example 18, the subject matter of any one or more of Examples 16-17 optionally include wherein the enlarged lens comprises a body having a shape selected from the group comprising a bulbous shape, a triangular shape, a square shape and a trapezoidal shape.

In Example 19, the subject matter of any one or more of Examples 1-18 optionally include a light generator coupled to the light conductor, the light generator comprising a laser module; and a controller for operating the light generator to produce a laser beam; wherein the controller is configured to generate cleaning laser pulses intermittently with fragmenting laser pulses, the cleaning laser pulses being of high power and short duration to remove debris attached to the device.

In Example 20, the subject matter of any one or more of Examples 1-19 optionally include wherein the shaft further comprises a working channel extending from the proximal portion to the distal portion.

Example 21 is a method of preventing damage to an optical fiber in a medical device having laser treatment capabilities, the method comprising: emitting a laser beam from the optical fiber; fragmenting a biological stone with the laser beam; and mitigating damage to the optical fiber from fragmentation of the biological stone.

In Example 22, the subject matter of Example 21 optionally includes wherein mitigating damage to the optical fiber from fragmentation of the biological stone comprises: reducing effects of reflected laser light on the optical fiber with a deflection lumen.

In Example 23, the subject matter of Example 22 optionally includes dissipating reflected laser light through the deflection lumen.

In Example 24, the subject matter of any one or more of Examples 22-23 optionally include absorbing reflected laser light within the deflection lumen.

In Example 25, the subject matter of any one or more of Examples 21-24 optionally include wherein mitigating damage to the optical fiber from fragmentation of the biological stone comprises: reflecting reflected laser light with a mirror.

In Example 26, the subject matter of Example 25 optionally includes passing the emitted laser beam through an optical device on which the mirror is located.

In Example 27, the subject matter of Example 26 optionally includes bending the laser beam with the optical device, wherein the optical device comprises a prism.

In Example 28, the subject matter of any one or more of Examples 21-27 optionally include wherein mitigating damage to the optical fiber from fragmentation of the biological stone comprises: absorbing reflected laser energy with a shield.

In Example 29, the subject matter of any one or more of Examples 21-28 optionally include wherein mitigating damage to the optical fiber from fragmentation of the biological stone comprises: absorbing kinetic energy from fragments of the biological stone with a shield.

In Example 30, the subject matter of any one or more of Examples 21-29 optionally include wherein mitigating damage to the optical fiber from fragmentation of the biological stone comprises: absorbing heat from fragments of the biological stone with a shield.

In Example 31, the subject matter of any one or more of Examples 21-30 optionally include wherein mitigating damage to the optical fiber from fragmentation of the biological stone comprises: shaping a shockwave generated by the laser beam in fluid disposed between the medical device and the biological stone.

In Example 32, the subject matter of Example 31 optionally includes wherein shaping a shockwave generated by the laser beam in fluid disposed between the medical device and the biological stone comprises: dispersing the laser beam exiting from the optical fiber at a distal end of the optical fiber.

In Example 33, the subject matter of Example 32 optionally includes wherein dispersing the laser beam exiting from the optical fiber at a distal end of the optical fiber comprises: passing the laser beam through an optical device located at a distal end of the optical fiber.

In Example 34, the subject matter of Example 33 optionally includes wherein passing the laser beam through an optical device located at the distal end of the optical fiber comprises passing the laser beam through an enlarged portion of the optical fiber having at least one surface oblique to a central axis of the optical fiber.

In Example 35, the subject matter of any one or more of Examples 33-34 optionally include wherein shaping a shockwave generated by the laser beam in fluid disposed between the medical device and the biological stone comprises: dispersing thermal energy density of the shockwave over a larger surface area of the biological stone compared to emitting the laser beam without the optical device.

In Example 36, the subject matter of any one or more of Examples 21-35 optionally include wherein mitigating damage to the optical fiber from fragmentation of the biological stone comprises: removing debris of the biological stone from the medical device with pulses of the laser beam, the pulses comprising short duration, high energy bursts of the laser beam.

In Example 37, the subject matter of Example 36 optionally includes wherein the pulses are at regular intervals determined by a control unit of a laser generator.

In Example 38, the subject matter of any one or more of Examples 36-37 optionally include wherein the pulses are at triggered as a result of sensing a drop in power output of a laser generator.

In Example 39, the subject matter of any one or more of Examples 36-38 optionally include wherein removing debris of the biological stone from the medical device with pulses of the laser beam comprises vaporizing dust of the biological stone.

In Example 40, the subject matter of any one or more of Examples 36-39 optionally include wherein removing debris of the biological stone from the medical device with pulses of the laser beam comprises displacing fragments of the biological stone.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A device for performing a surgical procedure, the device comprising:
a shaft extending from a proximal portion to a distal portion;
a light conductor extending at least partially through the shaft to be exposed at the distal portion; and
a damage mitigator positioned to receive light from the light conductor to discharge the light from the device,
wherein the damage mitigator is configured to protect the light conductor from incoming energy comprising at least one of laser energy, thermal energy and kinetic energy.

2. The device of claim 1, wherein:
the light conductor is pinned at a proximal location and at a distal location;
the shaft spans a first length between the proximal location and the distal location; and
the light conductor has a second length between the proximal location and the distal location that is greater than the first length to produce slack in the light conductor.

3. The device of claim 1, wherein:
the light conductor includes a distal end; and
the shaft comprises a distal end face including an opening to receive the distal end of the light conductor.

4. The device of claim 3, wherein the damage mitigator comprises a lumen extending into the distal end face to receive the distal end of the light conductor, wherein the distal end of the light conductor is positioned a distance from the distal end face of the shaft within the lumen.

5. The device of claim 4, wherein the light conductor has a first diameter and the lumen has a second diameter, wherein the second diameter is different than the first diameter and the distance is fixed.

6. The device of claim 1, wherein the damage mitigator comprises an optical device comprising:
a first side facing the light conductor; and
a second side facing away from the light conductor;
wherein the optical device is configured to allow light from the light conductor to pass through; and
wherein the optical device is configured to reflect light at the second side.

7. The device of claim 6, wherein the optical device comprises at least one of a prism and a mirror coating.

8. The device of claim 1, wherein the damage mitigator comprises a shield, the shield being formed of a material that is light transmitting and harder than the material of the light conductor, wherein the shield comprises a sapphire body and the light conductor comprises silica or quartz.

9. The device of claim 8, wherein the shield has a first diameter and the light conductor has a second diameter, wherein the second diameter is larger than the first diameter, wherein the shield is uncoupled from the light conductor.

10. The device of claim 8, wherein the shield comprises an anti-reflective coating.

11. The device of claim 1, wherein the damage mitigator comprises an enlarged lens located at a distal end of the light conductor, the enlarged lens including at least one surface that is non-parallel to a central axis of the light conductor, wherein the enlarged lens comprises an enlarged portion of a distal end of the light conductor.

12. The device of claim 1, wherein the damage mitigator comprises an enlarged lens located at a distal end of the light conductor, the enlarged lens including at least one surface that is non-parallel to a central axis of the light conductor, wherein the enlarged lens comprises a body having a shape selected from the group comprising a bulbous shape, a triangular shape, a square shape and a trapezoidal shape.

13. The device of claim 1, further comprising:
a light generator coupled to the light conductor, the light generator comprising a laser module; and
a controller for operating the light generator to produce a laser beam;
wherein the controller is configured to generate cleaning laser pulses intermittently with fragmenting laser pulses, the cleaning laser pulses being of high power and short duration to remove debris attached to the device; and
wherein the shaft further comprises a working channel extending from the proximal portion to the distal portion.

14. A method of preventing damage to an optical fiber in a medical device having laser treatment capabilities, the method comprising:
emitting a laser beam from the optical fiber;
fragmenting a biological stone with the laser beam; and
mitigating damage to the optical fiber from fragmentation of the biological stone.

15. The method of claim 14, wherein mitigating damage to the optical fiber from fragmentation of the biological stone comprises:
reducing effects of reflected laser light on the optical fiber with a deflection lumen by at least one of dissipating reflected laser light through the deflection lumen and absorbing reflected laser light within the deflection lumen.

16. The method of claim 14, wherein mitigating damage to the optical fiber from fragmentation of the biological stone comprises:
reflecting reflected laser light with a mirror;
passing the emitted laser beam through an optical device on which the mirror is located; and
bending the laser beam with the optical device, wherein the optical device comprises a prism.

17. The method of claim 14, wherein mitigating damage to the optical fiber from fragmentation of the biological stone comprises at least one of:
absorbing reflected laser energy with a shield;
absorbing kinetic energy from fragments of the biological stone with a shield; and
absorbing heat from fragments of the biological stone with a shield.

18. The method of claim 14, wherein mitigating damage to the optical fiber from fragmentation of the biological stone comprises:
shaping a shockwave generated by the laser beam in fluid disposed between the medical device and the biological stone;
dispersing the laser beam exiting from the optical fiber at a distal end of the optical fiber; and
passing the laser beam through an optical device located at a distal end of the optical fiber.

19. The method of claim 18, wherein:
passing the laser beam through an optical device located at the distal end of the optical fiber comprises passing the laser beam through an enlarged portion of the optical fiber having at least one surface oblique to a central axis of the optical fiber; and
shaping a shockwave generated by the laser beam in fluid disposed between the medical device and the biological stone comprises dispersing thermal energy density of the shockwave over a larger surface area of the biological stone compared to emitting the laser beam without the optical device.

20. The method of claim 14, wherein mitigating damage to the optical fiber from fragmentation of the biological stone comprises:
removing debris of the biological stone from the medical device with pulses of the laser beam, the pulses comprising short duration, high energy bursts of the laser beam.

21. The method of claim 20, wherein:
the pulses are at regular intervals determined by a control unit of a laser generator;
the pulses are at triggered as a result of sensing a drop in power output of a laser generator; and
removing debris of the biological stone from the medical device with pulses of the laser beam comprises at least one of vaporizing dust of the biological stone and displacing fragments of the biological stone.
